(19) United States
(12) Patent Application Publication
(10) Pub. No.: US 2010/0300694 A1
(43) Pub. Date: Dec. 2, 2010

(54) METHOD FOR PRODUCING AN ORGANIC COMPOSITION CONTAINING AN N-NONYL ETHER

(76) Inventors: Anja Vonderhagen, Dusseldorf (DE); Peter Daute, Beverstedt (DE)

Correspondence Address: SMITH MOORE LEATHERWOOD LLP, P.O. BOX 21927, GREENSBORO, NC, 27420, US

(21) Appl. No.: 12/743,850

(22) PCT Filed: Nov. 20, 2008
(86) PCT No.: PCT/EP08/65938

§ 371 Date: Jul. 22, 2010

(30) Foreign Application Priority Data
Nov. 20, 2007 (DE) 10 2007 055 594.8
Feb. 14, 2008 (DE) 10 2008 009 368.8

(51) Int. Cl. E21B 37/00; C07C 43/04; C07C 43/13; C11D 9/60; A61K 8/92; C11D 3/20; C08K 5/06; C09K 3/00; B01F 17/00; C09K 8/34; E21B 43/00

(52) U.S. Cl. 166/311; 568/671; 568/678; 510/392; 514/772; 510/188; 524/376; 516/116; 252/8.81; 106/287.24; 507/109; 426/302; 166/369

(57) ABSTRACT

The present invention relates to a method for producing an organic composition containing a functional component selected from the group consisting of a thermoplastic polymer, an enzyme, a setting agent, a paraffin, an oil, a colorant and a hair or skin care substance. The method includes providing an n-nonyl ether, a functional component which is able to react with an n-nonyl alcohol component, and, if appropriate, at least one further additive substance; and mixing the n-nonyl ether, the functional component and the at least one further additive substance. The invention further relates to a method for producing and using a molded article, a method for producing an item to be packaged, the use of at least one n-nonyl ether, a method for producing and/or cleaning the surfaces of boreholes, drilling devises or drill cuttings, and also to methods for producing an oil or a gas.

The present invention relates to a method for producing an organic composition containing a functional component selected from the group consisting of a thermoplastic polymer, an enzyme, a setting agent, a paraffin, an oil, a colorant and a hair or skin care substance, and also an n-nonyl ether, to a method for producing a molded article, to a method for producing an item to be packaged, to the use of at least one n-nonyl ether, to the use of a molded article, to a method for cleaning the surfaces of boreholes, drilling devices or drill cuttings, to methods for producing a borehole and also to methods for producing an oil or a gas.

Short to medium-chain linear fatty alcohols are nowadays successfully used as raw materials for surfactants, foam influencing agents, solvents, consistency-providing agents, lubricant additives and as an etherification or esterification component in the processing of plastics materials. Either linear C₈ or C₁₀ alcohols or branched C₉ alcohols (i-nonanol) are available. Linear alcohols are usually of native origin and always even-numbered. C₈/C₁₀ cuts comprising 40 to 48% by weight of C₈ alcohols and 51 to 59% by weight of C₉ alcohols are preferably used in this regard.

Although pure C₁₀ alcohol and the derivatives thereof, such as for example ether or ester, have a high boiling point and are thus comparatively involatile, they display high solidification points. Although pure C₈ alcohol and the derivatives thereof are, for their part, characterized by relatively low solidification points, they have low boiling points and are thus very volatile.

Branched i-nonanols are substance mixes and are produced petrochemically. The branching of alcohols leads to poorer biodegradability. A further drawback in relation to the use of i-nonanols is the excessively high melting point or the excessively low boiling range of the derivatives such as esters, ethoxylates, sulphates, even when alcohol mixes are used. The non-ideal viscosity behavior, in particular at relatively low temperatures, therefore imposes limits on this product group.

The present invention was based on the object of at least partially overcoming the drawbacks resulting from the prior art.

In particular, the present invention was based on the object of disclosing a method allowing organic compositions containing ethers of short to medium-chain linear fatty alcohols to be provided as an additive, wherein these organic compositions comprise fewer highly volatile components than the comparable organic compositions known in the art, and which display satisfactory viscosity behavior even at low temperatures.

In addition, the present invention was based on the object of disclosing a method allowing organic compositions containing ethers of short to medium-chain linear fatty alcohols to be provided as an additive, as many components as possible of these organic compositions being based on renewable raw materials or on starting materials which can be obtained from renewable raw materials.

In addition, the organic compositions which can be obtained using this method are to display improved, application-related properties compared to the organic compositions known in the art.

In particular, the present invention was based on the object of disclosing a compound which may in particular also be used as an additive in drilling fluids or cleaning agents for drilling devices.

A contribution to solving at least one of the objects mentioned hereinbefore is made by the subject matters of the generic claims, the sub-claims dependent thereon representing further embodiments according to the invention.

The present invention therefore relates in particular to a method for producing an organic composition comprising a functional component selected from the group consisting of a thermoplastic polymer, an enzyme, a setting agent, a paraffin, an oil, a colorant and a hair care or skin care substance, comprising as method steps:
i) providing
   ia) an n-nonyl ether as an additive that can be obtained by reacting an n-nonyl alcohol component with a further component which is able to react with the n-nonyl alcohol component so as to form an n-nonyl ether,
   ib) the functional component, and optionally
   ic) at least one further additive;
ii) mixing the n-nonyl ether, the functional component and optionally the at least one further additive.

The term a “functional component” refers in the sense of the present invention preferably to a component which imparts to the composition to which this functional component is added its characteristic, functional property. Thus, in the sense of the present invention, the functional component of a thermoplastic composition is the thermoplastic polymer, the functional component of an adhesive is the setting agent, the functional component of a lubricant formulation is the oil, the functional component of a washing agent is the enzyme, the functional component of a defoamer is the paraffin, the functional component of a paint or a dye is the colorant and the functional component of a cosmetic preparation is the hair or skin care substance.

The term an “organic composition” refers in the sense of the present invention preferably to a composition, more than 50% by weight of which, based on the total weight of the organic composition, consists of organic components, the term an “organic component” referring preferably to a carbon-containing compound except for CO₂, CO, carbides, CSO and pure carbon compounds such as graphite, carbon black or diamond. Preferably, the organic component is a hydrocarbon compound which can comprise oxygen, nitrogen, phosphorus, sulphur or at least two of these atoms as heteroatoms.

Step ia) of the method according to the invention firstly provides an n-nonyl ether as an additive which can be obtained by reacting an n-nonyl alcohol component with a further component which is able to react with the n-nonyl alcohol component so as to form an n-nonyl ether.

This provision of an n-nonyl ether preferably comprises the following method steps:
ia1) providing an n-nonyl alcohol component;
ia2) providing a further component which is able to react with the n-nonyl alcohol component so as to form an n-nonyl ether;
ia3) reacting the n-nonyl alcohol component with the at least one further component so as to form an n-nonyl ether.

In method step 1a1) of the method for providing an n-nonyl ether, an n-nonyl alcohol component is firstly provided. In accordance with a preferred embodiment of the method according to the invention for producing an organic composition, it is preferable for at least 80% by weight, particularly preferably at least 90% by weight and most preferably at least 99% by weight of the n-nonyl alcohol component, based in each case on the n-nonyl alcohol component provided, to be obtained from pelargonic acid. In this connection, it is furthermore preferable for the provision of the n-nonyl alcohol component to include the catalytic hydrogenation of pelargonic acid (octane carboxylic acid, nonanoic acid), for example according to the method described in WO-A-2006/021328, or else the catalytic hydrogenation of the oleic acid ozonide formed during the ozonolysis of oleic acid or both. Also conceivable is the catalytic hydrogenation of esters of pelargonic acid, for example the catalytic hydrogenation of the methyl, ethyl, propyl or butyl ester of pelargonic acid. If the n-nonyl alcohol component is obtained by the catalytic hydrogenation of pelargonic acid, then the pelargonic acid itself can for example be obtained by ozonolysis of oleic acid and subsequent, oxidative working-up of oleic acid ozonide or else by ozonolysis of erucic acid and subsequent oxidative working-up of erucic acid ozonide. A method of this type is carried out on a large scale by Unilever®, Emery® and Henkel®, for example, and is also described inter alia in “Ozonierung von Alkenen in Alkoholen als Lösungsmittel”, dissertation by Eberhard Rischbieter, Universität Carolo-Wilhelmina zu Braunschweig, 2000 or in U.S. Pat. No. 2,813,113. The oxidation of the aldehydes formed during the oxidative working-up of ozonides with the formation of the corresponding acid derivatives is for example described in DE-C-100 70 770. Oleic acid can in turn be produced from tallow or tall oils, such as is described in U.S. Pat. No. 6,498,261, for example. In addition to the ozonolysis of oleic acid or erucic acid, pelargonic acid can also be obtained by summarization of petrochemical raw materials. The petrochemical production of pelargonic acid is also conceivable, such as is described for example by Harold A., Wittcoff, Bryan G., Reuben, Jeffrey S. Plotkin in “Fats and Oils”, Industrial Organic Chemicals (Second Edition) (2004), John Wiley & Sons, Inc., pages 411-434, or else the production of pelargonic acid from oleic acid in accordance with the method described in GB-A-813842.

In accordance with a particular embodiment of the method according to the invention for producing an organic composition, the n-nonyl alcohol component used for producing the n-nonyl ether comprises, in addition to the n-nonyl alcohol, further alcohols, for example C₈ and/or C₁₀ alcohols, although it is in this case particularly preferable for the n-nonyl alcohol component to contain less than 10% by weight, particularly preferably less than 7.5% by weight and most preferably less than 5% by weight, based in each case on the n-nonyl alcohol component, of C₈ and C₁₀ alcohols. The n-nonyl alcohol content of the n-nonyl alcohol component is, in the case of a use of a mixture of n-nonyl alcohol and at least one further alcohol, preferably at least 90% by weight, particularly preferably at least 92.5% by weight and most preferably at least 95% by weight, based in each case on the total weight of the n-nonyl alcohol component.

An n-nonyl alcohol component which is particularly preferred in accordance with the invention is in particular that n-nonyl alcohol component which is obtained by catalytic hydrogenation of the pelargonic acid sold under the brand names EMERY°1202, EMERY°1203 and EMERY°1210, EMERY°1202 consisting for less than 1% by weight of C₆ monocarboxylic acids, for about 1% by weight of C₇ monocarboxylic acids, for about 4% by weight of C₈ monocarboxylic acids, for about 93% by weight of pelargonic acid and for about 2% by weight of other by-products, in particular monocarboxylic acids containing more than 9 carbon atoms, EMERY® 1203 consisting for about 0.1% by weight of C₆-C₈ monocarboxylic acids, for about 99% by weight of pelargonic acid and for about 0.9% by weight of other by-products, in particular monocarboxylic acids containing more than 9 carbon atoms and EMERY® 1210 consisting for about 3% by weight of C₅ monocarboxylic acids, for about 27% by weight of C₆ monocarboxylic acids, for about 31% by weight of C₇ monocarboxylic acids, for about 12% by weight of C₈ monocarboxylic acids and for about 27% by weight of pelargonic acid, although the use of EMERY® 1203 is particularly preferred, as the pelargonic acid content is particularly high in this case. Also advantageous in principle are n-nonyl alcohol components which were obtained by catalytic hydrogenation of pelargonic acid mixtures and comprise more than 10% by weight, particularly preferably more than 25% by weight of pelargonic acid.

In method step ia2) of the method for providing an n-nonyl ether at least one further component which is able to react with the n-nonyl alcohol component so as to form an n-nonyl ether is provided, this further component preferably being an alcohol, an epoxide, a halogen alkane or a mixture of at least two thereof.

In the case of an alcohol as the further component, it is preferable for this alcohol to be selected from the group consisting of C₁ to C₃₀ alkanols, particularly preferably C₁ to C₂₀ alkanols and most preferably C₁ to C₁₀ alkanols, such as for example methanol, ethanol, propanol, butanol, pentanol, hexanol, heptanol, octanol and nonanol, of C₁ to C₃₀ diols, particularly preferably of C₁ to C₂₀ diols and most preferably C₁ to C₁₀ diols, such as for example glycol and propanediol, of C₁ to C₃₀ triols, particularly preferably of C₁ to C₂₀ triols and most preferably C₁ to C₁₀ triols, such as for example glycerol, polyalcohols or polyether alcohols, such as for example diethylene glycol, dipropylene glycol, triethylene glycol, triethylene glycol, tetraethylene glycol, tetrapropylene glycol, polyethylene glycols having a molecular weight of more than 100 g/mol, polypropylene glycols having a molecular weight of more than 120 g/mol, polyethylene glycol, polypropylene glycol, pentaerythritol, multiple glycerides and saccharides, and also mixtures of at least two of the alcohols mentioned hereinbefore.

In the case of an epoxide as the further component, use is preferably made of epoxides of C₂ to C₂₀ hydrocarbons and particularly preferably of C₂ to C₁₄ hydrocarbons, ethylene oxide, propylene oxide and glycidol being particularly preferred epoxides and ethylene oxide and propylene oxide being the most preferred epoxides. Epoxystearic acid and diepoxylinoleic acid or the derivatives thereof are also possible as the further component.

In the case of a halogen alkane as the further component, possible alkanes are in particular chlorine alkanes, such as for example 1-chlorobutane, 2-chlorobutane, 1-chloropentane, 2-chloropentane or 3-chloropentane.

In method step ia3) of the method for providing an n-nonyl ether, the n-nonyl alcohol component is reacted with the at least one component so as to form an n-nonyl ether.

If the n-nonyl ether is produced by a condensation reaction between the alcohols in the n-nonyl alcohol component and the alcohols used as the further component, then the alcohols are condensed with dehydration preferably continuously, in particular in a fixed bed reactor which is charged with suitable catalysts, such as for example with alumina molded articles, in particular with γ-alumina, preferably in the form of pellets, tablets, extrudates, balls or granules, or else with zeolite-based catalyst systems. The condensation is preferably carried out at temperatures of from 200° C. to 260° C., particularly preferably from 220° C. to 260° C. and at a pressure of from 10 mbar to 60 bar. Depending on the temperature and pressure range, the condensation takes place in the gas and/or liquid phase. The optimum temperatures are dependent on the starting material(s) used, the progress of the reaction, the type of catalyst and the concentration of catalyst. They can easily be determined for each individual case by tests. Elevated temperatures increase the reaction speeds and promote secondary reactions, such as for example the elimination of water from alcohols or the formation of colored by-products. A suitable method for producing an ether using halogen alkane sulphonic acids as catalysts is described in DE-A-195 11 668, for example.

The crude product obtained in this way generally consists of a mix of starting material, olefins and dialkyl ethers which can be separated by distillation, for example, the non-reacted alcohol preferably being fed back into the process again. The method for producing the n-nonyl ether is preferably conducted at an LHSV (“liquid hour space velocity”=m³ of alcohol/(h×m³ of bulk volume of catalyst)) of from 0.2 to 1.4, if appropriate based on the introduction of liquid starting materials. The production of dialkyl ethers by the condensation of diols is described in DE-A-10 2004 056 786 or in WO-A-97/035823, for example, the disclosed content of which concerning the production of dialkyl ethers from alcohols is hereby incorporated by reference and forms part of the disclosure of the present invention.

If the n-nonyl ether is produced by a substitution reaction between the alcohols in the n-nonyl alcohol component and an epoxide, for example ethylene oxide or propylene oxide, as the further component, then reacting takes place, in this case too, preferably in the presence of suitable catalysts, such as for example zeolites or hydrophobized hydrotalcites. The reaction of ethylene oxide and propylene oxide with alcohols, for example, while forming multiply ethoxylated or multiply propoxylated ethers is described in DE-A-40 10 606, for example, the disclosed content of which concerning the production of dialkyl ethers from alcohols and ethylene oxide or propylene oxide is hereby incorporated by reference and forms part of the disclosure of the present invention.

In accordance with a particularly preferred embodiment of the method according to the invention for producing an organic composition, it is preferable for the n-nonyl ether provided in method step i) to be a polyether alcohol with 2 to 30 ether repeating units, particularly preferably with 4 to 20 ether repeating units, these ether repeating units being preferably an —[O—CH₂—CH₂] unit, an —[O—CH₂—CH₂—CH₂] unit or a mixture of these units. Polyether alcohols of this type can be obtained by reacting the n-nonyl alcohol component in a condensation reaction with a polyethylene glycol of corresponding chain length or by reacting the n-nonyl alcohol component in a substitution reaction with ethylene oxide, propylene oxide or a mixture of ethylene oxide and propylene oxide in relative amounts such that 2 to 30 ether repeating units, particularly preferably 4 to 20 ether repeating units are bound to the n-nonyl alcohol component.

In accordance with a further, preferred embodiment of the method according to the invention for producing an organic composition, it is preferable for the n-nonyl ether, but in particular the n-nonyl alcohol-based polyether alcohol described hereinbefore to be used as a modified n-nonyl ether in the form of an organic or inorganic ester. In this case, the method for providing an n-nonyl ether also comprises the further method step of:

1a4) esterifying the n-nonyl ether obtained in method step 1a3),

such esterification being possible only when a polyhydric alcohol or else an epoxide was used as the further component in method step 1a2), as only then are n-nonyl ethers obtained that still comprise free, esterifiable OH groups.

All organic and inorganic acids which are known to the person skilled in the art and are able to react with OH-functional, organic compounds can be used as the acid component for the esterification. The use of mono-, di- or polycarboxylic acids is particularly preferred in accordance with the invention. Examples of monocarboxylic acids which are suitable in this connection are acetic acid, butyric acid, acrylic acid, methacrylic acid, oleic acid, oxalic acid, stearic acid, succinic acid, citric acid, fumaric acid, maleic acid, benzoic acid or citric acid, whereas the dicarboxylic acids used may for example be oxalic acid, malonic acid, succinic acid, glutaric acid, adipic acid, pimelic acid, suberic acid, azelaic acid, sebacic acid, tartaric acid, malic acid, α-ketoglutaric acid, oxaloacetic acid, orthophthalic acid, isophthalic acid or terephthalic acid. An example of a suitable tricarboxylic acid is for example trimellitic acid. In addition to the organic acids mentioned hereinbefore, use may also be made of inorganic acids, such as for example sulphuric acid, phosphoric acid or boric acid, so that the corresponding sulphates, phosphates or borates are obtained. Sulphonic acids, such as for example benzene sulphonic acid, n-nonyl sulphonic acid, dodecyl benzene sulphonic acid, dodecyl benzene sulphonate, dodecyl benzene sulphonic acid, ammonium dodecyl benzene sulphonate, benzene sulphonic acid or dodecyl benzene sulphonic acid, can also be used as the acid. Also conceivable is the use of a mixture of at least two of the acid components mentioned hereinbefore, including in particular the use of a mixture of an organic and of an inorganic acid.

It is also preferable for the acid component to be reacted with the n-nonyl ether in a quantitative ratio such that the molar ratio of carboxylic acid groups:hydroxyl groups is in a range of from 1:1.0 to 1:5.0, particularly preferably in a range of from 1:1.2 to 1:2 and most preferably in a range of from 1:1.7 to 1:1.9.

The esterification takes place in this case preferably in the presence of an esterification catalyst. The esterification catalysts used may be acids, such as for example sulphuric acid or p-toluene sulphonic acid, or metals and the compounds thereof. Suitable examples are tin, titanium, zirconium, which are used as finely divided metals or expediently in the form of their salts, oxides or soluble organic compounds. In contrast to protonic acids, metal catalysts are high-temperature catalysts which generally reach their full activity only at temperatures above 180° C. They are however preferred in accordance with the invention because they produce fewer by-products, such as for example olefins, than proton catalysis. Esterification catalysts which are particularly preferred in accordance with the invention are one or more divalent tin compounds or tin compounds or elemental tin which can react with the starting materials to form divalent tin compounds. For example, the catalyst used may be tin, tin (II) chloride, tin (II) sulphate, tin (II) alcoholates or tin (II) salts of organic acids, in particular of mono- and dicarboxylic acids. Particularly preferred tin catalysts are tin (II) oxalate and tin (II) benzoate. The esterification reaction can be carried out by methods known to the person skilled in the art. It may be particularly advantageous in this regard to remove the water formed during the reaction from the reaction mix, this removal of water being carried out preferably by distillation, if appropriate by distillation with excess-used 1,2-propanediol. It is also preferable to carry out the esterification reaction at a temperature in a range of from 50 to 300° C., particularly preferably in a range of from 100 to 250° C. and most preferably in a range of from 150 to 200° C. In this case too, the optimum temperatures depend on the feedstock alcohol(s), the progress of the reaction, the type of catalyst and the concentration of catalyst and can easily be determined for each individual case by tests.

In method step ib) of the method according to the invention for producing an organic composition, a functional component is provided.
1. In accordance with a first variant of the method according to the invention, the functional component is a thermoplastic polymer and the organic composition is therefore a thermoplastic, organic composition.

The term “thermoplastic polymer”, such as it is used in the present document, refers to plastics materials which are easily (thermoplastically) deformable in a specific temperature range. This process is reversible and can be repeated as many times as desired by cooling and reheating into the molten state, provided that overheating does not cause thermal decomposition of the material.

The thermoplastic polymers which can be used as the functional component in accordance with the first variant of the method according to the invention are generally polycondensates or chain polymers or a mixture of these two, in particular thermoplastic polyurethanes, thermoplastic polyesters, thermoplastic polyamides, thermoplastic polyolefins, thermoplastic polyvinyl esters, thermoplastic polyethers, thermoplastic polystyrenes, thermoplastic polyimides, thermoplastic sulphur polymers, thermoplastic polyacetals, thermoplastic fluoroplastics, thermoplastic styrene-olefin copolymers, thermoplastic polyacrylates, thermoplastic ethylene-vinyl acetate copolymers or mixes of two or more of the thermoplastic polymers mentioned hereinbefore.

However, according to the invention, it is preferable for more than 90% by weight, particularly preferably more than 95% by weight, in addition even more preferably at least 99% by weight and most preferably 100% by weight of thermoplastic polymer, based in each case on the total weight of the thermoplastic polymer, to be based on thermoplastic polyesters. The term “polyester”, such as it is used in the present document, includes in particular polymers which were obtained by a polycondensation reaction between a polycarboxylic acid and a polyol (what are known as “AA//BB-polyesters”) or by a polycondensation reaction of a hydroxycarboxylic acid or by ring-opening polymerization of a cyclic ester (what are known as “AB-polyesters”). In one configuration according to the invention, polycarbonates obtainable by reacting phosgene with diols may be excluded from the term “polyester” as used in accordance with the invention.

In principle, all currently known thermoplastic polyesters and copolyesters may be used. Examples of polyesters of this type include substantially linear polyesters which were produced via a condensation reaction of at least one polycarboxylic acid, preferably a dicarboxylic acid (dibasic acid) or an ester-forming derivative thereof and at least one polyol, preferably a divalent alcohol (diol). The preferably dibasic acid and the preferably divalent diol may both be either aliphatic or aromatic, although aromatic and partially aromatic polyesters as thermoplastic molding materials are particularly preferred in view of their high softening points and hydrolytic stability. In aromatic polyesters, substantially all the ester links are attached to the aromatic rings. They may be semicrystalline and even display liquid-crystalline behavior or be amorphous. Partially aromatic polyesters which were obtained from at least one aromatic dicarboxylic acid or an ester-forming derivative thereof and at least one aliphatic diol are thermoplastic polyesters which are particularly preferred in accordance with the invention. Examples of suitable aromatic dicarboxylic acids include terephthalic acid, 1,4-naphthalene dicarboxylic acid or 4,4′-biphenyl dicarboxylic acid. Examples of suitable aliphatic diols include alkylene diols, especially those containing 2 to 6 C atoms, preferably 2 to 4 C atoms, particular examples of these being ethylene glycol, propylene diols and butylene diols. Preferably, ethylene glycol, 1,3-propylene diol or 1,4-butylene diol is used as the polyol or diol component for producing the thermoplastic polyesters contained in the composition according to the invention as component a). Thermoplastic polyesters which are particularly preferred in accordance with the invention and can be obtained by reacting a dicarboxylic acid with a diol include in particular polyalkylene terephthalates, for example polyethylene terephthalate (PET), polypropylene terephthalate (PPT) or polybutylene terephthalate (PBT), polyalkylene naphthalates, for example polyethylene naphthalate (PEN) or polybutylene naphthalate (PBN), polylactic acid (PLA), polyalkylene dibenzoates, for example polyethylene bibenzoate and also mixtures of at least two of these thermoplastic polyesters.

These partially aromatic polyesters described hereinbefore can optionally comprise a small quantity of units originating from other dicarboxylic acids, for example isophthalic acid, or other diols such as cyclohexanedimethanol; this generally reduces the melting point of the polyester. A special group of partially aromatic polyesters are what are known as segmented or block copolyesters which contain, in addition to the polyester segments mentioned hereinbefore (also referred to as “hard segments”), what are known as “soft segments”. These soft segments originate from a flexible polymer; that is to say a substantially amorphous polymer having a low glass transition temperature (T_{g}) and low rigidity, with reactive end groups, preferably two hydroxyl groups. The glass transition temperature of these “soft segments” is preferably below 0° C., particularly preferably below −20° C. and most preferably below −40° C. In principle, a plurality of different polymers can be used as the soft segment. Suitable examples of “soft segments” are aliphatic polyethers, aliphatic polyesters or aliphatic polycarbonates. The molar mass of the soft segments can vary widely, but is preferably between 400 and 6,000 g/mol.

In addition to the above-mentioned linear polyesters, which can be obtained via a polycondensation reaction of at least one polycarboxylic acid or an ester-forming derivative thereof and at least one polyol, the main component used in accordance with the first variant of the method according to the invention may also be thermoplastic polyesters which can be obtained by a polycondensation reaction of short-chain hydroxycarboxylic acids or by a ring-opening reaction of cyclic esters.

Examples of suitable, short-chain hydroxycarboxylic acids which can be used for producing thermoplastic polymers include in particular L-lactic acid, D-lactic acid, DL-lactic acid, glycolic acid, 3-hydroxybutyric acid, 4-hydroxybutyric acid, 4-hydroxyvaleric acid, 5-hydroxyvaleric acid, 6-hydroxycaproic acid and also mixtures of these hydroxycarboxylic acids. Examples of suitable cyclic esters include in particular glycolide (a dimer of glycolic acid) and ε-caprolactone (a cyclic ester of 6-hydroxycaproic acid).

The production of the thermoplastic polyesters described hereinbefore is also described inter alia in “Encyclopedia of Polymer Science and Engineering”, Volume 12, pages 1 to 75 and pages 217 to 256; John Wiley & Sons (1988) and also in “Ullmann's Encyclopedia of Industrial Chemistry”, Volume A21, pages 227 to 251, VCH Publishers Inc. (1992). Thermoplastic polymers which are preferred in accordance with the invention are polyethylene terephthalate (PET), polybutylene terephthalate (PBT) and polylactic acid (PLA). Furthermore, it is preferable in accordance with the first variant of the method according to the invention for this thermoplastic polymer to be able to be used as a functional component in an amount of at least 60% by weight, preferably of at least 75% by weight and particularly preferably of at least 90% by weight, based in each case on the total weight of the organic composition, whereas the n-nonyl ether is used as an additive, in particular as a mold release agent, as an antifogging agent, as a plasticizer, as an antistatic agent or as a lubricant, preferably in an amount in a range of from 0.001 to 40% by weight, particularly preferably in an amount in a range of from 0.01 to 25% by weight and most preferably in an amount in a range of from 0.1 to 10% by weight, based in each case on the total weight of the thermoplastic composition. If the n-nonyl ether is used as an additive in thermoplastic, organic compositions for the purposes mentioned hereinbefore, it is particularly preferable for the n-nonyl ether to be a polyalkylene glycol ether of the n-nonanol, particularly preferably a polyethylene glycol ether or polypropylene glycol ether of the n-nonanol and most preferably a polyethylene glycol ether of the n-nonanol, the polyalkylene glycol which was used to produce the polyalkylene glycol ether preferably having a molecular weight of more than 100 g/mol.

Examples of additives which can be provided in method step ic) in accordance with this first variant of the method according to the invention are in particular impact modifiers, filler materials, reinforcing agents, flame retardant compounds, heat and UV stabilizers, antioxidants, other processing aids, nucleating agents, dyestuffs and antidrip agents. Examples of suitable impact modifiers, filler materials, reinforcing agents and flame retardant compounds may be inferred inter alia from US 2005/0234171 A1. These further additives are used preferably in an amount in a range of from 0.001 to 20% by weight, particularly preferably in an amount in a range of from 0.01 to 10% by weight and most preferably in an amount in a range of from 0.1 to 5% by weight, based in each case on the total weight of the thermoplastic composition.

The mixing, which in the case of the first variant of the method according to the invention takes place in method step ii), of the n-nonyl ether, the functional component (thermoplastic polymer) and if appropriate the additive can be carried out using known techniques. Thus, the mixing may for example be a dry mixing process in which the various components are mixed below the melt processing temperature of the thermoplastic polymer, or else a melt mixing method in which the components are if appropriate premixed and mixed at the melt processing temperatures of the thermoplastic polymer. The melt mixing methods include in particular the melt kneading method which is preferred in accordance with the invention and can be implemented, for example, by continuous melt kneading using a single-screw kneading machine, a twin-screw kneading machine of the toothing-same-direction-of-rotation type, toothing-different-directions-of-rotation type, non-toothing-same-direction-of-rotation type, non-toothing-different-directions-of-rotation type or of other types, or by batch melt kneading using a roller kneading machine, a Banbury kneading machine or the like. A combination of a dry mixing method and a melt mixing method is also conceivable.

Furthermore, the order and the nature of the addition of the individual components ia), ib) and if appropriate ic) into the mixing device are in principle non-critical. Thus, for example, the thermoplastic polymer and if appropriate the additive substances can firstly be placed in the mixing device and the n-nonyl ether be added only subsequently. It is also conceivable firstly to mix the n-nonyl ether or a part of the n-nonyl ether with one or more other components of the thermoplastic composition according to the invention, for example with one or more additive substances, and then either to add this mixture to the thermoplastic polymer which is already contained in the mixing device or else firstly to place this mixture in the mixing device and only then to add the thermoplastic polymer.

In further configurations of the method according to the invention for producing an organic composition in accordance with the first variant of the method according to the invention, the mixing is carried out in accordance with at least one of the following measures:
M1) at the glass transition temperature of the thermoplastic polymer or at a temperature above the glass transition temperature of the thermoplastic polymer;
M2) the n-nonyl ether being more liquid than thermoplastic polymer; or
M3) at least a part of the n-nonyl ether being added to the progenitor of the thermoplastic polymer.

Furthermore, it is in keeping with configurations according to the invention if two or more of the foregoing measures are combined. Specifically, as configurations, this produces the following combinations of measures illustrated based on the combinations of figures: M1M2, M1M3, M2M3 and M1M2M3.

In accordance with a preferred embodiment M1 of the method according to the invention, the components provided in method steps ia), ib) and if appropriate ic) are mixed in method step ii) of the method according to the invention by a melt mixing method. In this connection, it is particularly preferable for the mixing in method step ii) to be carried out at the glass transition temperature of the thermoplastic polymer or at a temperature above the glass transition temperature of the thermoplastic polymer. It is particularly preferable in this connection for the mixing to be carried out at a temperature in a range of from 5 degrees below the glass transition temperature (T_{g}) to 200° C. above the glass transition temperature of the thermoplastic polymer used, particularly preferably at a temperature in a range of from 1 degree below the glass transition temperature (T_{g}) to 180° C. above the glass transition temperature of the thermoplastic polymer used and most preferably at a temperature in a range of from 1 degree above the glass transition temperature (T_{g}) to 150° C. above the glass transition temperature of the thermoplastic polymer used, although the upper limit of the temperature range is delimited substantially by the decomposition temperature of the thermoplastic polymer used. Furthermore, it is in keeping with configurations according to the invention if the mixing is carried out at temperatures in a range of from 10 to 180° C. and preferably 50 to 150° C. above the glass transition temperature of the thermoplastic polymer used.

In configuration M2 according to the invention, in which the n-nonyl ether is more liquid than the thermoplastic polymer, it is preferable to use the n-nonyl ether at a temperature at which said n-nonyl ether is liquid and the thermoplastic polymer is not yet liquid. Preferably, the temperature of the thermoplastic polymer is in this case below the glass transition temperature of this polymer. Thus, it is preferable if the melting temperature of the n-nonyl ether and the glass transition temperature of the thermoplastic polymer differ by at least 5° C., preferably at least 10° C. and particularly preferably at least 30° C. Furthermore, it is preferable in this configuration and also generally to use the thermoplastic polymer as granules. Generally speaking, all granule forms known to the person skilled in the art, with a spherical or cylindrical three-dimensional shape, are possible in this case too. The granule size, which is determined by means of sieve analysis, is for at least 70% by weight of the granular particles in a range of from 0.01 to 5 cm and preferably in a range of from 0.1 to 4 cm. The procedure according to this configuration allows the surfaces of the granular particles to be coated at least partly with the n-nonyl ether, so that at least partially coated thermoplastic polymer granules are obtained. This allows the n-nonyl ether according to the invention to be distributed as homogeneously as possible in the thermoplastic composition, in particular when said composition is prepared as a formulation for the subsequent extrusion.

In configuration M3 according to the invention, in which the n-nonyl ether is added to the progenitor of the thermoplastic polymer, the n-nonyl ether may be either in liquid or in solid form. The progenitor of the thermoplastic polymer may in principle be in the form of all precursors known to the person skilled in the art before the thermoplastic polymer is obtained. These include in particular precursors having a lower molecular weight than the final thermoplastic polymer. In this case, it is preferable for the molecular weight of the progenitor to differ from that of the finished thermoplastic polymer by a factor of at least 1.1, preferably at least 1.5 and particularly preferably at least by a factor of 2. In addition to the monomers and oligomers, which preferably consist of 2 to 100 monomers, used to produce the thermoplastic polymer, a further component, in particular with polycondensates, is a prepolymer which is completely polymerized, usually by heat treatment, to form the finished thermoplastic polymer. Preferably, the prepolymer is based on more than 100 monomers as repeating units, wherein the number of monomers as repeating units, and thus the final molecular weight of the finished thermoplastic polymer, is not achieved. Thus, it is particularly preferable to add the n-nonyl ether in each case to the monomers, oligomers or the prepolymer or at least two of these. This allows, in addition to a homogeneous distribution of the n-nonyl ether, also an incorporation, usually as a result of the conditions prevailing during the polymerization or complete polymerization, of the n-nonyl ether as a result of chemical bonds with the thermoplastic polymer.
2. In accordance with a second variant of the method according to the invention, the functional component is an enzyme and the organic composition is a washing agent.

Suitable enzymes are in particular amylases, proteases, lipases, cellulases, peroxidases or mixtures of at least two of these enzymes.

Amylases are added to remove starch and glycogen. Alpha-, beta- and gamma-amylases and also glucoamylases and maltogenic amylases can be used in accordance with the invention. Suitable amylases are commercially available under the names Duramyl®, Termamyl®, Fungamyl® and BAN® (Novo Nordisk), and also Maxamyl®, or Purafect® OxAm, for example. The amylases can originate from any desired sources, such as for example from bacteria, fungi, pancreas glands of animal origin, from germinated cereals or from yeast. Even genetically modified amylases can be used, if appropriate even preferably, as the functional component in the organic compositions according to the invention. The compositions according to the invention can contain the amylase enzymes in an amount of from 0.0001% by weight to 5% by weight, particularly preferably from 0.0001% by weight to 1% by weight and most preferably from 0.0005 to 0.5% by weight, based in each case on the total weight of the organic composition.

In accordance with the second variant of the method according to the invention, in addition to amylases, proteases can also be added to the organic compositions according to the invention for the cleavage of proteins and peptide residues. Proteases are particularly suitable for the hydrolytic cleavage and removal of protein residues, in particular dried-on protein residues. Proteases which are suitable in accordance with the invention are proteinases (endopeptidases) and peptidases (exopeptidases). Proteases which can be used may be of vegetable, animal, bacterial and/or fungal origin. Suitable proteases are in particular serine, cysteine, aspartate and metal proteases. Even genetically modified proteases can be used, if appropriate even preferably, in the compositions according to the invention. Proteases which can be used are commercially available under the names Alcalase®, BLAP®, Durazym®, Esperase®, Everlase®, Maxapem®, Maxatase®, Optimase Purafect® OxP or Savinase®. Conventionally, proteases are used in an amount of from 0.00001 to 1.5% by weight and particularly preferably of from 0.0001 to 0.75% by weight, based in each case on the total weight of the organic composition.

In accordance with the second variant of the method according to the invention lipases can also be used as the functional component. They serve to remove tightly clinging fatty soil. Lipases are thus a biological alternative to surfactants and can assist the cleaning effect of surfactants in a range of from 0.0001 to 1% by weight, based on the total weight of the organic composition. Suitable lipases may be obtained from plants (for example ricinus species), microorganisms and animal sources, such as for example pancreatic lipases. Commercially available lipases are for example Lipolase®, Lipomax®, Lipozym® and Lumafast®.

The enzymes mentioned hereinbefore can if appropriate be combined with any other desired enzymes in order to further improve the cleaning performance of the organic composition used as a washing agent. Further enzymes which are suitable in accordance with the invention are cellulases, hemicellulases, peroxidases, reductases, oxidases, ligninases, cutinases, pectinases, xylanases, phenoloxidases, lipoxygenases, tannases, pentosanases, malanases, glucanases, arabinosidases and any desired mixtures of these enzymes.

In this second variant of the method according to the invention, the n-nonyl ether is added to the washing agent preferably in the function of a surfactant, wherein it is in this case preferable for the n-nonyl ether to be used in an amount of from 0.001 to 40% by weight, particularly preferably from 0.01 to 30% by weight, even more preferably from 0.1 to 20% by weight and most preferably from 1 to 10% by weight, based in each case on the total weight of the organic composition.

Examples of additives which can be provided, in accordance with this second variant of the method according to the invention, in method step ic) are in particular further surfactants differing from n-nonyl ether, builders, solvents, hydrophobic components, phase separation aids, thickening agents, polymers, soil-release active ingredients, solubilisers, hydrotropes, such as for example sodium cumene sulphonate, octyl sulphate, butyl glucoside, butyl glycol, emulsifiers, such as for example bile soap, shine drying additives, cleaning enhancers, antimicrobial active ingredients or disinfectants, antistatics, preservatives, such as for example glutaraldehyde, bleaching systems, perfumes, fragrances, dyestuffs, opacifiers or else skin protecting agents, the amount of additives of this type conventionally being not greater than 12% by weight, based on the total weight of the organic composition.

An overview of the additives contained in washing agents, of the amounts in which said additives are added to the washing agents and also of the manner in which a washing agent is produced from the components mentioned hereinbefore, in the sense of method step ii) of the method according to the invention for producing an organic composition, may be inferred inter alia from DE 101 06 712 A1.
3. In accordance with a third variant of the method according to the invention, the functional component is a setting agent and the organic composition is an adhesive.

The chemical composition of the setting agent contained in the adhesive depends on the manner in which the setting is carried out by the adhesive. Thus, it may be a physically setting adhesive, for example a hot-melt adhesives containing for example ethylene-vinyl acetate copolymers, polyamides or polyesters as the setting agent, a solvent-containing wet adhesive, containing for example polymeric vinyl compounds, polymethyl methacrylate or natural and synthetic rubber as the setting agent, a contact adhesive, containing for example polychloroprenes or butadiene acrylonitrile rubber as the setting agent, a dispersion adhesive, containing for example polyvinyl acetate, vinyl acetate copolymers, polyacrylates, polyvinylidene chloride, styrene-butadiene copolymers, polyurethanes, polychloroprene or rubber latices as the setting agent, a water-based adhesive, containing for example glutine glues, such as for example hide glue or fish glue, glues based on natural vegetable products, such as for example starch glue, methylcellulose or casein glue, or polyvinyl alcohol adhesives as the setting agent, a pressure-sensitive adhesive, containing for example polyacrylates, polyvinyl ethers or natural rubber as the setting agent, or a plastisol, containing for example PVC and plasticizer as the setting agent.

Furthermore, the adhesive may be a chemically curing adhesive, for example a cyanoacrylate-based adhesive, containing for example cyanoacrylic acid esters as the setting agent, a methyl methacrylate-based adhesive, containing for example methacrylic acid methyl esters as the setting agent, an anaerobically curing adhesive, containing for example diacrylic acid esters of diols as the setting agent, a radiation-curable adhesive, containing for example epoxy acrylates or polyester acrylates as the setting agent, a phenol formaldehyde resin-based adhesive, containing for example phenols and formaldehyde as the setting agent, a silicone-based adhesive, containing for example polyorganosiloxanes as the setting agent, a polyimide-based adhesive, containing for example aromatic tetracarboxylic acid anhydrides and aromatic diamines as the setting agent, an epoxy resin adhesive, containing for example oligomeric diepoxides and polyamines or polyamidoamines as the setting agent, or a polyurethane-based adhesive, containing for example di- and if appropriate trifunctional isocyanates and polyols as the setting agent.

The concentration of setting agent in the adhesive is dependent on the type of adhesive used, but is conventionally in a range of from 10 to 100% by weight, particularly preferably from 20 to 90% by weight and additionally preferably from 30 to 80% by weight, based in each case on the total weight of the adhesive.

In this third variant of the method according to the invention, the n-nonyl ether is added to the adhesive preferably in the function of a solvent, a consistency-providing agent or else in the function of a surfactant, wherein it is in this case preferable for the n-nonyl ether to be used in an amount of from 0.001 to 40% by weight, particularly preferably from 0.1 to 30% by weight, even more preferably from 1 to 20% by weight and most preferably from 3 to 10% by weight, based in each case on the total weight of the organic composition. In particular if the n-nonyl ether is used as a solvent, for example in solvent-containing wet adhesives, the concentration of the n-nonyl ether may if appropriate even be above the concentration ranges mentioned hereinbefore.

The additives, which in accordance with this third variant of the method according to the invention can be provided in method step ic), depend on the nature of the respective adhesive. Particular examples include fillers, such as for example chalks, natural ground or precipitated calcium carbonates, calcium magnesium carbonates (dolomite), silicates such as for example aluminum silicates, heavy spar or magnesium aluminum silicates, talc and also reinforcing fillers such as for example carbon blacks, in particular lamp blacks, channel blacks, gas blacks, furnace blacks or mixtures thereof, plasticizers or else plasticizer mixtures, catalysts (in the case of chemically setting adhesives), stabilizers and also solvents. The amount of additives of this type is dependent on the type of the respective additive and is conventionally not greater than 50% by weight, based on the total weight of the organic composition.

An overview of adhesives is provided inter alia by the publication “Kleben/Klebstoffe” from the Fonds der Chemischen Industrie im Verband der Chemischen Industrie e.V. (Chemical Industry Fund within the German Chemical Industry Association), 2001.
4. In accordance with a fourth variant of the method according to the invention, the functional component is a paraffin, in particular a paraffin wax, and the organic composition is a defoamer.

The paraffin, which in the fourth variant of the method according to the invention is provided as the functional component in method step ib), is generally a complex substance mix without a clear melting point. It is characterized conventionally by defining its melting range by differential thermal analysis (DTA), as described in “The Analyst” (1962), 420, and/or its solidification point. This refers to the temperature at which the wax passes from the liquid to the solid state as a result of slow cooling. Paraffins containing less than 17 C atoms cannot be used in accordance with the invention; their content in the paraffin wax mix should therefore be as low as possible and is preferably below the limit which can be significantly measured using conventional analytical methods, for example gas chromatography. Preferably, use is made of waxes which solidify in the range from 20° C. to 70° C. In this case, it should be borne in mind that even paraffin wax mixes which appear solid at room temperature can contain different contents of liquid paraffin. In the paraffin waxes which can be used in accordance with the invention, the liquid content is as high as possible at 40° C. without having yet reached 100% at this temperature. Particularly preferred paraffin wax mixes have at 40° C. a liquid content of at least 50% by weight, in particular of from 55% by weight to 80% by weight, and at 60° C. a liquid content of at least 90% by weight. As a result, the paraffins are free-flowing and pumpable at temperatures down to at least 70° C., preferably down to at least 60° C. It should also be borne in mind that the paraffins contain no volatile contents where possible. Preferred paraffin waxes contain less than 1% by weight, in particular less than 0.5% by weight of vaporable contents at 110° C. and normal pressure. Paraffin waxes which can be used in accordance with the invention can for example be purchased under the commercial names Lunaflex® from the company Fuller and also Deawax® from DEA Mineralöl AG. The amount of paraffin in the organic composition acting as the defoamer is preferably in a range of from 50 to 99% by weight, particularly preferably from 60 to 95% by weight and most preferably from 70 to 95% by weight, based in each case on the total weight of the organic composition. If, however, excipients are added to the defoamer, then the paraffin content may also be well below the concentration ranges mentioned hereinbefore.

In this fourth variant of the method according to the invention, the n-nonyl ether is added to the defoamer preferably in the function of a solvent or else in the function of a surfactant, wherein it is in this case preferable for the n-nonyl ether to be used in an amount of from 0.001 to 20% by weight, particularly preferably from 0.1 to 10% by weight, even more preferably from 1 to 8% by weight and most preferably from 2 to 7% by weight, based in each case on the total weight of the organic composition.

The additives, which in accordance with this fourth variant of the method according to the invention can be provided in method step ic), may for example be silicone oils and the blends thereof with hydrophobized silica, or further compounds having a defoaming effect, such as for example bisamides. The defoamer can also contain excipients which preferably have a granular structure and consist of water-soluble or water-dispersible, surfactant-free compounds, in particular of inorganic and/or organic salts which are suitable for use in washing and cleaning agents. Examples of water-soluble, inorganic excipients include in particular alkali carbonate, alkali borate, alkali aluminosilicate and/or alkali sulphate, whereas the organic excipients which can be used are for example acetates, tartrates, succinates, citrates, carboxymethyl succinates and also the alkali salts of aminopolycarboxylic acids, such as EDTA, hydroxyalkane phosphonates and aminoalkane polyphosphonates, such as 1-hydroxyethane-1,1-diphosphonate, ethylene diamine tetramethylene phosphonate and diethylene triamine pentamethylene phosphonate. The use of film-forming polymers, such as for example polyethylene glycols, polyvinyl alcohols, polyvinylpyrrolidones, polyacrylates and cellulose derivatives, as excipients is also conceivable. The amount of additives of this type is conventionally not greater than 25% by weight, based on the total weight of the organic composition. However, excipients can also be used in a much higher concentration.

Examples of a defoamer which can be produced in accordance with the fourth variant of the method according to the invention include the defoamers mentioned in WO-A-1997/034983, wherein the disclosed content of said document concerning the method for producing a defoamer from the components provided in method steps 1a, 1b and 1c), in the sense of method step ii), is in particular hereby also incorporated by reference and forms part of the disclosure of the present invention.
5. In accordance with a fifth variant of the method according to the invention, the functional component is an oil, preferably a hydrocarbon containing 20 to 35 carbon atoms (lubricating oil), and the organic composition is a lubricant formulation.

The oil contained in the lubricant formulation may be a raffinate which was obtained by separating off the hydrocarbons which are naturally present in crude oil and contain 20 to 35 carbon atoms, a hydrocrack oil (HC synthetic oil) which was obtained by cracking crude oil constituents comprising more than 35 carbon atoms, or else synthetic hydrocarbons which are obtained by cracking crude oil constituents containing less than 12 carbon atoms to form gases, such as in particular ethene or butene, and the subsequent synthesis of hydrocarbons containing 20 to 35 carbon atoms from these gases.

In addition to these oils, the lubricant formulation can also contain bio oils obtained from renewable raw materials, wherein bio oils from the HETG, HEPG, HEPR or the HEES group (VDMA 24568 ISO Standard 15380) can in particular be used. The HETG group includes triglycerides, such as for example rape oil, whereas the HEPG group includes polyglycols. The HEES group includes synthetic esters, in particular TMP esters (trimethylpropane esters, also referred to as oleic acid ester or trioleate). The HEPR group includes liquids consisting for the most part of polyalphaolefins (PAOs) and related hydrocarbons.

The amount of oil in the lubricant formulation is preferably in a range of from 50 to 99% by weight, particularly preferably in a range of from 60 to 95% by weight and most preferably in a range of from 70 to 90% by weight, based in each case on the total weight of the organic composition. If, however, the lubricant formulation is also to be used for cooling, then it can also comprise large amounts of water, wherein the oil content of the lubricant formulation can in this case also be well below the concentration ranges mentioned hereinbefore.

In this fifth variant of the method according to the invention, the n-nonyl ether is added to the lubricant formulation preferably in the function of a solvent or else in the function of a surfactant, wherein it is in this case preferable for the n-nonyl ether to be used in an amount of from 0.001 to 40% by weight, particularly preferably from 0.1 to 30% by weight, even more preferably from 1 to 20% by weight and most preferably from 2 to 10% by weight, based in each case on the total weight of the organic composition.

The additives, which in accordance with this fifth variant of the method according to the invention can be provided in method step ic), may in particular be surface-active, oil-improving or oil-protecting additives. The surface-active additives include detergents, dispersants, high pressure or wear-protecting, corrosion and rust-preventing and also coefficient of friction-altering additives. The oil-improving additives alter the properties of the oil with regard to viscosity, the pour point and in relation to the elastomers of seals, for example. The oil-protecting additives protect the oil from aging, deactivate metal particles and prevent foaming of the oil. Even very finely ground solids, such as for example Teflon® (PTFE), ceramic oxides or molybdenum disulphide compounds, can be added as an additive. If the lubricant composition is also to be used as a coolant, then it can additionally contain water in amounts of up to 95% by weight, particularly preferably in amounts of up to 90% by weight, the lubricant composition preferably being in the form of an emulsion in such a case.
6. In accordance with a sixth variant of the method according to the invention, the functional component is a colorant and the organic composition is a paint or a dye. In this case, the term a “dye” refers in the sense of the present invention to a non-shiny, open-pored coating having a high dyestuff and pigment content, but only a low binder content, whereas the term a “paint” refers to a composition for coating surfaces made of wood, metal, plastics material or mineral material, which composition has a higher binder content than a dye.

The colorant may be an inorganic or an organic colorant, wherein these colorants may be water-soluble or water-insoluble. Particular examples include inorganic or organic, preferably powdered pigments. Pigments differ from dyestuffs in so far as they are insoluble in their application media. Suitable inorganic pigments and the manners in which they are produced may be inferred from G. Buxbaum; “Industrial Inorganic Pigments”, 1^{st} ed., pp. 85-107; VCH Verlagsgesellschaft mbH, Weinheim, 1993, G. Buxbaum; “Industrial Inorganic Pigments”, 1^{st} ed., pp. 114-117; VCH Verlagsgesellschaft mbH, Weinheim, 1993 and also G. Buxbaum; “Industrial Inorganic Pigments”, 1^{st} ed., pp. 124-131; VCH Verlagsgesellschaft mbH, Weinheim, 1993. The disclosure of said documents concerning inorganic pigments is hereby incorporated by reference and forms part of the disclosure of the present invention. Suitable organic pigments and the manner in which they are produced may be inferred in particular from W. Herbst and K. Hunger; “Industrielle organische Pigmente”; 2^{nd} ed.; pp. 4-11; VCH Verlagsgesellschaft mbH, Weinheim, 1995, W. Herbst and K. Hunger; “Industrielle organische Pigmente”; 2^{nd} ed.; p. 462; VCH Verlagsgesellschaft mbH, Weinheim, 1995, W. Herbst and K. Hunger; “Industrielle organische Pigmente”; 2^{nd} ed.; pp. 482-485; VCH Verlagsgesellschaft mbH, Weinheim, 1995, W. Herbst and K. Hunger; “Industrielle organische Pigment”; 2^{nd} ed.; p. 503; VCH Verlagsgesellschaft mbH, Weinheim, 1995 and also W. Herbst and K. Hunger; “Industrielle organische Pigmente”; 2^{nd} ed.; pp. 567-569; VCH Verlagsgesellschaft mbH, Weinheim, 1995. Examples of suitable classes of organic colorants (based on the basic element of the colored structural unit) include nitroso, nitro, monoazo, disazo, trisazo, stilbene, diphenylmethane, triarylmethane, xanthene, acridine, quinoline, thiazole, indamine, azine, oxazine, thiazine, lactone, phthalocyanine colorants.

These colorants can be contained in the paint or in the dye in amounts in a range of from 0.001 to 40% by weight, particularly preferably in amounts in a range of from 0.01 to 30% by weight, even more preferably in amounts in a range of from 0.01 to 30% [sic] by weight and most preferably in amounts in a range of from 0.1 to 10% by weight, based in each case on the total weight of the organic composition.

In this sixth variant of the method according to the invention, the n-nonyl ether is added to the paint or the dye preferably in the function of a solvent or else in the function of a surfactant, wherein it is in this case preferable for the n-nonyl ether to be used in an amount of from 0.001 to 40% by weight, particularly preferably from 0.1to 30% by weight, even more preferably from 1 to 20% by weight and most preferably from 2 to 10% by weight, based in each case on the total weight of the organic composition.

The additives, which in accordance with this sixth variant of the method according to the invention can be provided in method step ic), may in particular be binders, such as for example vegetable oils, balsamic resin from conifers, casein from milk, alkyd resin, polyurethane resin or epoxy resin, solvents, such as for example water, ethanol, citrus peel oil, white spirit, water or glycol ether, thixotropic agents, antioxidants, viscosity regulators, skinning and foam inhibitors, flow control agents, UV absorbers, extenders, preservatives or binders. The amount of the additives to be used may fluctuate widely. This applies in particular to the binders and solvents, depending on whether they are a paint or a dye.
7. In accordance with a seventh variant of the method according to the invention, the functional component is a hair care or skin care substance and the organic composition is a cosmetic preparation.

Examples of hair and/or skin care substances include in particular 18-β-glycyrrhetinic acid from liquorice root extract (Glycyrrhiza glabra), preferably at a purity of >99% of pure substance in the extract, aescin in horse chestnut (Aesculus hippocastanum), allantoin, aloe vera (containing mainly sugar, anthraquinone and minerals such as zinc), amino acids such as for example alanine, arginine, serine, lysine, ammonium glycyrrhizate from liquorice root extract, preferably at a purity of almost 100% of pure substance in the extract, apigenin from camomile extract (Matricaria recutita), arnica, in particular arnica montana or arnica chamissonis, asiaticoside and madecassoside in the Centella asiatica extract, avenanthramide from oat extract (Avena sativa), avocadol, azulene from camomile extract (Matricaria recutita), biotin (vitamin H), bisabolol from camomile extract (Matricaria recutita), brown algae extract (Ascophyllum nodosum), chlorogenic acid in water extract of Japanese honeysuckle (Lonicera japonica), coenzyme Q10, creatine, dexpanthenol, disodium glycyrrhizate from liquorice root extract, preferably at a purity of almost 100% of pure substance in the extract, extract from red algae (Asparagopsis armata), flavonoids from birch extract (Betula alba), flavonoids, vitexin in the extract of the passion flower (Passiflora incarnata), flavonoids, vitexin in the lime extract (Tilia platyphyllos), ginkgoflavonglycosides and terpene lactones in the ginkgo extract (Ginkgo biloba), ginsenosides in the ginseng extract (Panax ginseng), glycogen, grapefruit extract, hamamelis extract from virginian witch hazel (Hamamelis virginiana), honey, isoflavone glycosides in the clover extract (Trifolium pratense), St. John's wort extract from St. John's wort (Hypericum perforatum), jojoba oil, lecithin, maize oil (Zea mays), evening primrose oil, niacinamide, oenotheins B in the extract from willowherb (Epilobium angustifolium), oleuropein in the olive extract (Olea europaea), phytocohesine (sodium-beta-sitosterol sulphate), plankton extract (Tetraselmis suecica, Spirulina and others), polyphenols, catechins from the extract of grape seeds (Vitis vinifera), polyphenols, catechins from green tea (Camellia sinensis), marigold extract (Calendula officinalis), rosmarinic acid in melissa extract (Melissa officinalis), sea buckthorn oil, β-glucanes from oats (Avena sativa), stearyl glycyrrhetinic acid (stearyl esters of 18-β-beta glycyrrhetinic acid), sterols, sitosterol in the stinging nettle extract (Urtica dioica), sweet almond oil (Prunus dulcis), vitamin C and the esters thereof, vitamin E and the esters thereof, wheat germ oil zinc gluconate/magnesium aspartate/copper gluconate, and also zinc sulphate or zinc oxide and also proteins or protein derivatives, such as for example protein hydrolysates (for example collagen, keratin, silk protein or wheat protein hydrolysates).

These hair and/or skin care substances can be comprised in the cosmetic preparations in amounts in a range of from 0.001 to 40% by weight, particularly preferably in amounts in a range of from 0.01 to 30% by weight, even more preferably in amounts in a range of from 0.01 to 30% by weight and most preferably in amounts in a range of from 0.1 to 10% by weight, based in each case on the total weight of the organic composition.

In this seventh variant of the method according to the invention, the n-nonyl ether is added to the cosmetic preparation preferably in the function of a solvent or else in the function of a surfactant, wherein it is in this case preferable for the n-nonyl ether to be used in an amount of from 0.001 to 40% by weight, particularly preferably from 0.1 to 30% by weight, even more preferably from 1 to 20% by weight and most preferably from 2 to 10% by weight, based in each case on the total weight of the organic composition.

Suitable additive substances, which in accordance with this seventh variant of the method according to the invention can be provided in method step ic), may for example be inferred from Schrader, K., “Grundlagen and Rezepturen der Kosmetika”, 2^{nd} edition, 1989, pages 728-737, Domsch, A., “Die kosmetischen Präparate”, Verlag für chemische Industrie (H. Ziolkowsky, Ed.), 4^{th} edition, Volume 2, pages 212-230, 1992 or Johnson, D. H., “Hair and Hair Care”, New York, 1997, pages 65-104. The additive substances can be used in the conventional amounts known to the person skilled in the art, in particular in amounts of from 0.1 to 10.0% by weight, based on the total weight of the organic composition.

A contribution to achieving the objects mentioned at the outset is also made by a method for producing a shaped article, comprising the method steps:
I) providing a thermoplastic composition which can be obtained using the method described hereinbefore according to the first variant;
II) heating the thermoplastic composition to the glass transition temperature of the thermoplastic polymer or to a temperature above the glass transition temperature of the thermoplastic polymer;
III) producing a shaped article from the heated, thermoplastic composition produced in method step II).

In step I) of the method according to the invention for producing a shaped article, a thermoplastic composition according to the invention is firstly provided, this provision being carried out preferably by a method in accordance with a first variant of the method according to the invention.

Then, in method step II), the thermoplastic composition is heated to the glass transition temperature of the thermoplastic polymer or to a temperature above the glass transition temperature of the thermoplastic polymer. In this connection, it is again preferable for the thermoplastic composition to be heated to a temperature in a range of from 5 degrees below the glass transition temperature (T_{g}) to 100° C. above the glass transition temperature of the thermoplastic polymer used, particularly preferably to a temperature in a range of from 1 degree below the glass transition temperature (T_{g}) to 50° C. above the glass transition temperature of the thermoplastic polymer used and most preferably to a temperature in a range of from 1 degree above the glass transition temperature (T_{g}) to 20° C. above the glass transition temperature of the thermoplastic polymer used, although here too the upper limit of the temperature range is delimited substantially by the decomposition temperature of the thermoplastic polymer used.

In principle, method steps I) and II) can be carried out simultaneously or successively. Simultaneous carrying-out of method steps I) and II) is for example beneficial when the thermoplastic composition is produced by means of a melt mixing method. In this case, it may if appropriate be advantageous to transfer the composition produced by the melt mixing method directly to a shaped article. Successive carrying-out of method steps I) and II) is for example beneficial when the thermoplastic composition is produced by means of a dry mixing method or else when the thermoplastic composition is produced by means of a melt mixing method, but is not subjected to the formation of a molded article immediately after production; on the contrary, it is firstly cooled in accordance with method step v).

In method step III) of the method according to the invention for producing a shaped article, a shaped article is produced from the heated, thermoplastic composition produced in method step II). Particular examples of the method for producing a shaped article are injection molding, extrusion molding, compression molding, layered molding, lamination molding, hollow molding, vacuum molding and transfer molding, injection molding being particularly preferred.

Furthermore, it is in keeping with a configuration of the method according to the invention for producing a thermoplastic shaped article for, in at least one further method step IV), at least a partial region of the shaped article obtained in method step III) to serve as a shaped article blank and to be reduced in its mass cross section in relation to that of the shaped article obtained in method step III). The mass cross section is the cross section of a region of the shaped article that is made all the way through the thermoplastic molding material according to the invention. For example in receptacles or containers, the mass cross section is the thickness of a wall of these receptacles or containers. In shaped articles which are embodied in a more thread or cord-shaped manner, the mass cross section is the thickness of these threads or cords. In more planar formations such as plates, layers, webs, films or foils, the mass cross section is the thickness of these planar formations. For reducing the mass cross section, use may in principle be made of all suitable methods known to the person skilled in the art for this purpose. Examples of these include stretching in one or two directions, drawing in one or two directions, centrifuging or blow molding, which are each carried out preferably at elevated temperatures at which the thermoplastic composition according to the invention is sufficiently soft or even liquid to allow stretching, drawing, centrifuging or blow molding to be carried out. The partial region in which the cross section is reduced constitutes preferably at least 50% and particularly preferably at least 80% of the molded article obtained in step III). Generally speaking, stretching or drawing is carried out when a fiber is to be obtained from the shaped article obtained in step III). In the production of foils, on the one hand, the drawing or stretching can be carried out in one or more dimensions. Thus, the web issuing from an extruder can be drawn onto a roll at a higher speed than the exit speed from the extruder. If, on the other hand, a receptacle or container is to be obtained, then, apart from the stretching, drawing and centrifuging, above all blow molding is used in step IV). In this case, the mass cross section is reduced by applying a gas pressure. The gas pressure is generally selected in such a way as to allow the thermoplastic composition, which is usually heated at least to glass transition temperature, of the molded article obtained in step III) to be elongated. Generally speaking, the elongation is delimited as a result of the use of a mold having the end shape of the molded article. In this way, it is possible to produce, in addition to receptacles such as freezer compartments, trays and packagings for food products such as fruit, vegetables or meat, as well as pharmaceutical compositions as tablets, capsules, suppositories or powders, also containers for liquids. These liquid containers can be used not only for liquids of the cosmetic or pharmaceutical industry, but also in the food industry, preferably in the drinks industry, as multiple-use containers such as PET or PLA bottles. It is also possible for two or more of method steps I) to IV) to be supplemented by further method steps and/or to proceed at least with a time overlap. This applies in particular to method steps III) and IV).

Furthermore, the invention also allows other shaped articles apart from bottles to be produced. These include single and multiple-use containers such as plates, trays, pots or cups, and cutlery such as knives, forks or spoons. The biodegradable thermoplastic compositions according to the invention are particularly suitable for these applications.

A contribution to achieving the objects mentioned at the outset is also made by a method for producing an item to be packaged comprising as method steps:
a) providing an item and a shaped article, in particular a foil, wherein the shaped article can be obtained by the method described hereinbefore;
b) at least partially surrounding the item with the shaped article.

The item provided in method step a) is preferably a pharmaceutical, a body care product, an auxiliary agricultural agent, an adhesive, a building material, a dyestuff or a food product.

The method described in DE-A-103 56 769 can for example be used to at least partially surround the item.

A contribution to achieving the objects mentioned at the outset is also made by a method for coating substances which can be consumed by living beings, comprising as method steps:
A) providing a substance which can be consumed by living beings, for example a food product or pharmaceutical composition, and also an n-nonyl ether which can be obtained by reacting an n-nonyl alcohol component with a further component which is able to react with the n-nonyl alcohol component so as to form an n-nonyl ether;
B) at least partially surrounding the substance which can be consumed by living beings with the n-nonyl ether.

The n-nonyl ether is provided preferably in accordance with method step ia) of the method described at the outset for producing an organic composition.

The substance which can be consumed by living beings can for example be at least partially surrounded with the n-nonyl ether in such a way that the consumable substance and the n-nonyl ether are mixed together in suitable mixing devices, particular examples of mixing devices being the Patterson-Kelley mixer, DRAIS® turbulence mixer, Lödige® mixer, Ruberg® mixer, screw mixer, plate mixer and fluidized bed mixer and also continuously operating perpendicular mixers in which the polymer formation is mixed at rapid frequency by means of rotating blades (Schugi® mixer). Should the n-nonyl ether not be liquid under the mixing conditions, then this component must be heated to a temperature above the melting temperature of the n-nonyl ether before or during the mixing with the substance which can be consumed by living beings. In addition to the use of the mixing devices described hereinbefore, the substance which can be consumed by living beings can also be at least partially surrounded with the n-nonyl ether in that, for example, the substance which can be consumed by living beings is placed in a fluidized bed mixer and the n-nonyl ether is sprayed in liquid form onto the substance which can be consumed by living beings.

A contribution to achieving the objects mentioned at the outset is also made by the use of at least one n-nonyl ether which can be obtained by reacting an n-nonyl alcohol component with a further component which is able to react with the n-nonyl alcohol component so as to form an n-nonyl ether, as an additive in a composition containing as a functional component
α) a thermoplastic polymer, wherein the composition is a thermoplastic composition;
β) an enzyme, wherein the composition is a washing agent;
γ) a setting agent of an adhesive, wherein the composition is an adhesive;
δ) a paraffin, wherein the composition is a defoamer;
ε) an oil, wherein the composition is a lubricant formulation;
ζ) a colorant, wherein the composition is a paint or a dye; or
η) a hair care or skin care substance, wherein the composition is a cosmetic preparation,
   wherein the n-nonyl ether was obtained preferably by the method described at the outset for producing an n-nonyl ether, comprising method steps ia1), ia2), ia3) and optionally ia4).

A contribution to achieving the objects mentioned at the outset is also made by the use of the n-nonyl ether described at the outset, which can be obtained by reacting an n-nonyl alcohol component with a further component which is able to react with the n-nonyl alcohol component so as to form an n-nonyl ether, as an additive in compositions used in the drilling of boreholes.

It is particularly preferable in accordance with the invention for the n-nonyl ether described hereinbefore to be used as an additive in drilling fluids or cleaning agents for drilling devices.

The invention therefore also relates to a method for cleaning the surfaces of boreholes, in particular the walls of boreholes, of conveyor pipes or casings or of walls of the casing, and also for cleaning drilling devices or drill cuttings, wherein the surfaces are firstly brought into contact with a cleaning agent comprising the n-nonyl ether described hereinbefore and optionally the surfaces are subsequently rinsed down with water.

In this connection, it is particularly preferable for the cleaning agent to be used in the form of an aqueous solution, an aqueous dispersion or an oil-in-water emulsion containing
(α1) 0.1 to 50% by weight, particularly preferably 0.5 to 35% by weight, more preferably 1.0 to 15% by weight and most preferably 1.2 to 10% by weight of the n-nonyl ether,
(α2) 0 to 50% by weight, particularly preferably 0.5 to 35% by weight, more preferably 1.0 to 15% by weight and most preferably 1.2 to 10% by weight of further additives different from the n-nonyl ether, and also
(α3) 1 to 99.9% by weight, particularly preferably 30 to 99% by weight, more preferably 70 to 98% by weight and most preferably 80 to 97.6% by weight of water,
   the sum of components (α1) to (α3) being 100% by weight.

In particular, the amount of component (α1) in the aqueous composition may vary and is adapted to the type and the extent of the soiling.

Particular examples of additive (α2), which is different from the n-nonyl ether, are weighting agents, fluid-loss additives, viscosity-regulating additives, wetting agents or salts. The general regulations for the composition of the respective treatment liquids apply here.

The concomitant use of organic polymer compounds of natural and/or synthetic origin may also prove advantageous. Particular examples of this are starch or chemically modified starches, cellulose derivatives such as carboxymethylcellulose, guar gum, xanthan gum or else purely synthetic water-soluble and/or water-dispersible polymer compounds, in particular of the high-molecular polyacrylamide compounds type with or without anionic or cationic modification.

The term “drilling devices” includes in particular drilling implements, such as for example the drill tower, the drill string, in particular the drill rod assembly and the drill bit, cleaning installations, a solids disposal installation, in particular shaking screens or centrifuges, pumps, motors or gear mechanisms, or else the drilling platform or parts thereof. For cleaning the drilling devices, the cleaning agent containing the n-nonyl ether is sprayed onto or applied to the surfaces of the articles, or the articles to be cleaned are dipped into the aqueous compositions. At this point, the dirt becomes detached from the surfaces. Subsequently, the surfaces are brought into contact with water in such a way that the agents are removed together with the dirt, for example in that the surface is sprayed down using a jet of water.

Furthermore, the cleaning agent comprising the n-nonyl ether may be used to clean drill cuttings (or “cuttings” for short). Such cuttings are formed during drilling and have to be deposited, during off-shore drilling, onto the sea floor in the region surrounding the drilling platform; this can lead to a marked introduction of mineral oil into the environment. In order to substantially avoid ecological pollution of the sea, the cuttings are cleaned beforehand and the residues of the drilling fluid are removed therefrom. The cleaning agent comprising the n-nonyl ether can be used for all the cleaning processes with which the person skilled in the art is familiar and which occur in the field of ground drilling, both during off-shore drilling and when drilling on-shore. These include in particular the removal of paraffin deposits from borehole walls. Conventionally, boreholes are cleaned by pumping a pressurized cleaning liquid through the borehole, and the cleaning agent removes the deposits from the walls of the borehole. Subsequently, the dirt is transported with the liquid out of the borehole.

According to a preferred embodiment of the method according to the invention described hereinbefore, this comprises the method steps
(β1) drilling a borehole into the ground by means of a drill head driven via a drill rod assembly,
(β2) introducing a casing into the borehole, and
(β3) introducing cement into at least a partial region of the intermediate space between the outer side of the casing and the walls of the borehole,
   wherein, before method step (β3) is carried out, the cleaning agent comprising the n-nonyl ether is passed through the intermediate space between the outer side of the casing and the walls of the borehole, preferably circulated in this intermediate space. This circulating can for example be carried out in that the cleaning agent is pumped downward through the casing, preferably via the drill rod assembly, issues at the lower end of the casing, preferably on the drill head or on the drill bit, and then rises back up again through the intermediate space between the outer side of the casing and the walls of the borehole. If the cleaning agent is continuously pumped downward through the casing, both the walls of the borehole and the outer side of the casing can be cleaned in this way.

In accordance with a preferred embodiment of the method according to the invention for cleaning the surfaces of drilling devices, the method comprises the method step of drilling a borehole into the ground by means of a drill head driven via a drill rod assembly, wherein the cleaning agent containing the n-nonyl ether is at least partially passed through the drill head, preferably circulated at least partially there through, this passing-through or this circulating being carried out at least partially while the drill head is present in the borehole.

Particular examples of drilling devices, the surface of which can be cleaned using the cleaning agent, are, again, drilling implements, such as for example the drill tower, the drill string, in particular the drill rod assembly and the drill bit, cleaning installations, a solids disposal installation, in particular shaking screens or centrifuges, pumps, motors or gear mechanisms, or else the drilling platform or parts thereof.

A contribution to achieving the objects mentioned at the outset is also made by a method for producing a borehole, comprising the method steps
(β1) drilling a borehole into the ground by means of a drill head driven via a drill rod assembly,
(β2) introducing a casing into the borehole,
(β3) introducing cement into at least a partial region of the intermediate space between the outer side of the casing and the walls of the borehole,
(β4) optionally introducing a conveyor pipe into the casing,
(β5) optionally introducing a sealing liquid into the intermediate space between the outer side of the conveyor pipe and that of the inner side of the casing,
   wherein surfaces of the borehole, the guide pipe, the drill rod assembly or the drill head are brought into contact with the cleaning agent comprising the n-nonyl ether. In particular, this bringing-into-contact can be carried out in accordance with the preferred embodiments described hereinbefore of the method according to the invention for cleaning the surfaces of boreholes or drilling devices. It is accordingly preferable, before carrying out method step (β3), for the cleaning agent comprising the n-nonyl ether to be fed through the intermediate space between the outer side of the casing and the walls of the borehole, preferably to be circulated through this intermediate space.

As the sealing liquid which is introduced in method step (β5) into the intermediate space between the outer side of the conveyor pipe and that of the inner side of the casing, use may be made of all materials which are known for this purpose to the person skilled in the art. Those sealing liquids which are described in U.S. Pat. No. 7,219,735 may be mentioned at this point as an example.

A further contribution for achieving the objects mentioned at the outset is also made by a method for producing an oil or a gas that comprises, in addition to the aforementioned method steps (β1) to (β3) and if appropriate (β4) and (β5), also the method steps
(β6) conveying oil or gas through the borehole, and also
(β7) purifying or refining the conveyed oil or gas,
   the surfaces of the borehole, the conveyor pipe, the drill rod assembly or the drill head being in this case too brought into contact with the cleaning agent comprising the n-nonyl ether. In this case too, this bringing-into-contact can be carried out in accordance with the preferred embodiments described hereinbefore of the method according to the invention for cleaning the surfaces of boreholes or drilling implements.

The invention also relates to a method for producing boreholes, in which a drilling fluid is pumped through a borehole, a composition comprising the n-nonyl ether described at the outset being used as the drilling fluid.

According to a particular embodiment of this method, this composition is a water-in-oil emulsion.

In this connection, it is particularly preferable for the composition to contain
I) 28.9 to 99% by weight, particularly preferably 60 to 90% by weight and most preferably 70 to 80% by weight, based in each case on the total weight of the composition, of an organic oil phase which is not miscible with water,
II) 1 to 48% by weight, preferably, particularly preferably 5 to 40% by weight and most preferably 10 to 30% by weight, based in each case on the total weight of the composition, of water or aqueous phase,
III) 0.1 to 20% by weight, particularly preferably 1 to 15% by weight and most preferably 5 to 10% by weight, based in each case on the total weight of the composition, of the n-nonyl ether described at the outset, and also
IV) 0 to 70% by weight, particularly preferably 1 to 5% by weight and most preferably 1.5 to 3% by weight, based in each case on the total weight of the composition, of at least one further additive,
   the sum of components I) to IV) being 100% by weight.

In connection with the water-in-oil emulsion described hereinbefore, it is preferable for the organic oil phase I) to be selected wholly or partially from the group of the
a) paraffins containing 5 to 22 C atoms and/or
b) paraffins containing 5 to 22 C atoms and/or
c) internal olefins containing 12 to 30 C atoms in the molecule and/or
d) carboxylic acid esters of general formula R—COO—R, in which R represents a linear or branched, saturated or unsaturated alkyl radical containing 15 to 25 C atoms and R′ represents a saturated, linear or branched alkyl radical containing 3 to 22 C atoms, and/or
e) mineral oils, and/or
f) linear alpha-olefins (LAOS) containing 12 to 30 C atoms, and/or
g) carbonates.

In this connection, it is furthermore preferable for this water-in-oil emulsion to display a density of the liquid component in a range of from 1.2 to 3.0 g/cm³ and in particular in a range of from 1.5 to 3.0 g/cm³. The oil phase of the systems according to the invention contains components a) to e) alone or components a), b), d) or e) jointly blended with esters c) and also optionally blended with other suitable oil phases. Any desired mixtures of oil phases a) to e) with one another are also possible.

Component a)

According to the invention, linear or branched paraffins with 5 to 22 C atoms are used as component a). Paraffins—referred to more correctly as alkanes—are known to be saturated hydrocarbons which follow, for the linear or branched representatives, the general total formula CₙH₂ₙ₊₁. The cyclic alkanes follow the general total formula CₙH₂ₙ. The linear and branched paraffins are particularly preferred, whereas cyclic paraffins are less preferred. The use of branched paraffins is particularly preferred. Furthermore, preference is given to paraffins of the type that are liquid at room temperature, i.e. those containing 5 to 16 C atoms per molecule. However, it may also be preferable to use paraffins containing 17 to 22 C atoms, which display a wax-like consistency. However, it is preferable to use mixtures of the various paraffins, it being particularly preferable if these mixtures are still liquid at 21° C. Such mixtures can be formed, for example, from paraffins containing 10 to 21 C atoms. Paraffins are particularly preferred oil phases—alone or as a constituent of a mixture with further oil phases—in drilling fluids—preferably those of the invert type, in which the glycerol or oligoglycerol esters crosslinked in accordance with the invention are used as thickeners.

Component b)

Internal olefins (referred to hereinafter as IOs for short) can be used in accordance with the invention as component b). In this regard, IOs are likewise compounds which are known per se and can be produced by all the methods known to the person skilled in the art for this purpose. EP 0 787 706 A1 describes, for example, a method for synthesizing IOs by the isomerization of alpha-olefins on sulphonic or persulphonic acids. A characteristic feature of this is the fact that the IOs obtained in this way are linear and contain at least one olefinic double bond which is not in the alpha-position of the alkyl chain. Preferably, according to the invention, use is made of IOs or IO mixes of the type containing IOs containing 12 to 30 C atoms in the molecule, preferably containing 14 to 24 C atoms and in particular containing up to 20 C atoms in the molecule.

Component c)

Furthermore, esters of general formula R—COO—R′, in which R represents a linear or branched, saturated or unsaturated alkyl radical containing 15 to 25 C atoms and R′ represents a saturated, linear or branched alkyl radical containing 6 to 22 C atoms, are a constituent of the oil phases according to the invention. Even esters of this type are known chemical compounds. The basic use thereof in drilling fluids is, for example, the subject matter of EP 0 374 672 A1 or EP 0 374 671 A1. Particular preference is given to the use of esters of the type of which the radical R represents a saturated or unsaturated alkyl radical containing 15 to 25 and R′ represents a saturated alkyl radical containing 3 to 10 C atoms. The saturated compounds are particularly preferred in this regard. Within the scope of the inventive teaching, it is preferable for the oil phase to contain, in addition to the esters described hereinbefore, at most 15% by weight (based on the oil phase) of other esters comprising radicals R which represent alkyl radicals containing more than 23 C atoms.

Component d)

Mineral oils are a generic name for the liquid distillation products which consist substantially of mixes of saturated hydrocarbons and are obtained from mineral raw materials (crude oil, brown and hard coal, wood or peat). Preferably, the mineral oils contain only small quantities of aromatic hydrocarbons, preferably less than 3% by weight. Crude oil-based mineral oils which are liquid at 21° C. are preferred. The mineral oils preferably have boiling points of from 180 to 300° C.

Component e)

Linear alpha-olefins (or LAOs for short) are unsaturated hydrocarbons which are unbranched in the 1-position (“alpha-C atom”). They may be based on natural substances, but are in particular to a large extent also obtained synthetically. Natural substance-based LAOs are obtained by dehydration of natural substance-based fatty alcohols as linear products having a straight-chain carbon number. Even the synthetically obtained LAOs—produced by oligomerization of ethylene—frequently contain straight-chain carbon numbers in the chain, although methods are nowadays also known for producing odd-numbered alpha-olefins. In the sense of the definition according to the invention, they generally comprise—on account of their volatility—at least 10, preferably at least 12 to 14 C atoms in the molecule. The upper limit of the LAOS, which are free-flowing at room temperature, is in the range of from C₁₈ to C₂₀. However, this upper limit does not restrict the applicability of this class of substances within the scope of the invention. The upper limit of suitable LAO compounds for use within the scope of the teaching according to the invention is therefore well above the aforementioned limit value of C₁₈ to C₂₀ and may reach C₃₀, for example.

Component f)

The term “carbonates” refers, within the scope of the present application, to carbonic acid esters of fatty alcohols containing 8 to 22 C atoms, preferably the diesters of carbonic acid. Compounds of this type and the use thereof as an oil phase for drilling fluid are described in DE 40 18 228 A1.

In addition to components a) to f), the oil phase I) can contain still other, water-insoluble constituents, provided that these are ecologically compatible. Further particularly suitable mixture constituents of the oil phase I) according to the invention are therefore specifically:
(i) esters of C₁₋₅ monocarboxylic acids and monofunctional and/or polyfunctional alcohols, wherein radicals from monohydric alcohols comprises at least 6, preferably at least 8 C atoms and the polyhydric alcohols preferably have 2 to 6 C atoms in the molecule,
(ii) mixtures of secondary esters, selected from the group of propyl carboxylate, butyl carboxylate, pentyl carboxylate, hexyl carboxylate, heptyl carboxylate, octyl carboxylate, nonyl carboxylates, decyl carboxylate, undecyl carboxylate, dodecyl carboxylate, tridecyl carboxylate, tetradecyl carboxylate, pentadecyl carboxylate, hexadecyl carboxylate, heptadecyl carboxylate, octadecyl carboxylate, nonadecyl carboxylate, eicosyl carboxylate, uneicosyl carboxylate, doeicosyl carboxylate and isomers thereof, the secondary esters each comprising a carboxylate radical containing 1 to 5 C atoms, water-insoluble ethers of monohydric alcohols containing 6 to 24 C atoms,
(iii) water-insoluble alcohols containing 8 to 36 C atoms
(iv) polyalphaolefins (PAO)
(v) mixtures of components (i) to (iv)

The oil phase I) of the composition used as a drilling fluid in the form of a water-in-oil emulsion preferably has pour points below 0° C., preferably below −5° C. (measured in accordance with DIN ISO 3016: 1982-10). The Brookfield viscosity of the oil phase is at most 50 mPas at 0° C. The compositions used as the drilling fluid display, in so far as they are in the form of a W/O-type oil-based drilling fluid, a plastic viscosity (PV) in the range of from 10 to 70 mPas and a yield point (YP) of from 5 to 60 lb/100 ft², determined in each case at 50° C. The kinematic viscosity of the oil phase, measured in accordance with Ubbelohde at 20° C., should preferably be at most 12 mm²/sec. The aqueous phase of the agents according to the invention preferably has a pH value in the range of from 7.5 to 12, preferably from 7.5 to 11 and in particular from 8 to 10.

As the aqueous phase according to component II), the composition, which is used as the drilling fluid, preferably contains aqueous saline solutions, preferably saturated saline solutions, wherein the salts used may be all the alkali or alkaline earth halides known to the person skilled in the art. Particular examples of suitable salts include KCl, NaCl, LiCl, KBr, NaBr, LiBr, CaCl₂, and MgCl₂, wherein, of these, CaCl₂, NaCl and KCl or mixtures of these salts are particularly preferred.

Particular examples of further additives which can be contained, in accordance with component IV), in the composition used as the drilling fluid are additives selected from the group consisting of surfactants as an added component for crosslinked glycerol or oligoglycerol ester, weighting agents, fluid-loss additives, pH modifiers, further viscosity-modifying additives, wetting agents, salts, biocides, agents for inhibiting the undesirable exchange of water between drilled formations—for example water-swellable clays and/or salt layers—and, for example water-based, rinsing liquid, wetting agents for improved absorption of the emulsified oil phase on solid surfaces, for example for improving the lubricating effect, but also for improving the oleophilic closure of exposed rock formations, or rock faces, corrosion inhibitors, alkali reserves and emulsifiers.

The general regulations for the composition of the respective treatment liquids, for which exemplary indications are made hereinafter based on corresponding drilling muds, apply here. The additives may be water-soluble, oil-soluble and/or water or oil-dispersible.

The surfactants used may be anionic, nonionic, zwitterionic or cationic surfactants. However, the nonionic and the anionic surfactants are preferred. Typical examples of anionic surfactants are soaps, alkyl benzene sulphonates, alkane sulphonates, olefin sulphonates, alkyl ether sulphonates, glyceryl ether sulphonates, methyl ester sulphonates, sulpho fatty acids, alkyl sulphates, fatty alcohol ether sulphates, glycerol ether sulphates, fatty acid ether sulphates, hydroxy mixed ether sulphates, monoglyceride(ether)sulphates, fatty acid amide(ether)sulphates, mono- and dialkyl sulphosuccinates, mono- and dialkyl sulphosuccinamates, sulphotriglycerides, amide soaps, ether carboxylic acids and the salts thereof. The latter are particularly preferred surfactant components in the sense of the present technical teaching. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, if appropriate partially oxidized alk(en)yl oligoglycosides or glucoronic acid derivatives, fatty acid-N-alkyl glucamides, polyol fatty acid esters, sugar esters, sorbitan esters, polysorbates and amine oxides. In so far as the nonionic surfactants contain polyglycol ether chains, the chains may have a conventional, but preferably a narrowed distribution of homologues. The surfactants are an optional constituent in the additives. They are used preferably in amounts of from 0.01 to 2% by weight, in particular from 0.1 to 1.5% by weight and preferably from 0.2 to 0.5% by weight, based in each case on the total water-in-oil emulsion.

The emulsifiers are preferably nonionic emulsifiers which are in particular to be assigned to one of the following classes of substance: (oligo)alkoxylates—in particular low alkoxylates, corresponding ethoxylates and/or propoxylates being particularly important here—of basic molecules, which contain lipophilic radicals and are capable of alkoxylation, of natural and/or synthetic origin. Alkoxylates of the indicated type are known to be per se—i.e. with a terminal free hydroxyl group on the alkoxylate radical—nonionic emulsifiers; however, the corresponding compounds can also be end-capped, for example by esterification and/or etherification. A further important class of nonionic emulsifiers for the purposes of the invention are partial esters and/or partial ethers of polyfunctional alcohols containing in particular 2 to 6 C atoms and 2 to 6 OH groups and/or the oligomers thereof with acids and/or alcohols containing lipophilic radicals. Compounds of this type, which additionally contain bound into their molecular structure (oligo)alkoxy radicals and, in this case, in particular corresponding oligoethoxy radicals, are also particularly suitable in this regard. The polyfunctional alcohols containing 2 to 6 OH groups in the basic molecule or the oligomers derived therefrom may in particular be diols and/or triols or the oligomerization products thereof, wherein glycol and glycerol or the oligomers thereof may be particularly important. Known nonionic emulsifiers of the ethylene oxide/propylene oxide/butylene oxide block polymer type may also be assigned to the field of partial ethers of polyfunctional alcohols. A further example of corresponding emulsifier components are alkyl(poly)glycosides of long-chain alcohols and also the previously mentioned fatty alcohols of natural and/or synthetic origin or alkylolamides, amine oxides and lecithins. The concomitant use of alkyl(poly)glycoside compounds (APG compounds), which are nowadays conventional in the trade, as emulsifier components in the sense according to the invention may be particularly beneficial inter alia because this is a class of emulsifier of particularly pronounced ecological compatibility. In addition, reference may be made, without claim of completeness, to the following representatives of the substance classes listed in the present document of suitable emulsifier components: (oligo)alkoxylates of fatty alcohols, fatty acids, fatty amines, fatty amides, fatty acid and/or fatty alcohol esters and/or ethers, alkanolamides, alkylphenols and/or the reaction products thereof with formaldehyde and also further reaction products of carrier molecules containing lipophilic radicals with lower alkoxides. As indicated, the respective reaction products can also be at least partially end-capped. Examples of partial esters and/or partial ethers of polyfunctional alcohols are in particular the corresponding partial esters with fatty acids, for example of the glycerol monoester and/or diester type, glycol monoesters, corresponding partial esters of oligomerised polyfunctional alcohols, sorbitan partial esters and the like and also corresponding compounds with ether groupings.

Even the concomitant use of organic polymer compounds of natural and/or synthetic origin as further additives may be very important in this connection. Particular examples of this are starch or chemically modified starches, cellulose derivatives such as carboxymethylcellulose, guar gum, xanthan gum or else purely synthetic water-soluble and/or water-dispersible polymer compounds, in particular of the high-molecular polyacrylamide compounds with or without anionic or cationic modification type. Diluents for regulating viscosity: The aforementioned diluents may be organic or inorganic in nature; examples of organic diluents are tannins and/or quebracho extract. Further examples of this are lignite and lignite derivatives, in particular lignosulphonates.

Organophilic lignite is particularly preferred as an agent for preventing loss of liquid (fluid-loss additive), whereas preferred pH modifiers may be inferred from EP 0 382 701 A1, for example. The invention described in EP 0 382 701 A1 is based on the finding that use is to be made, in ester-based drilling fluids of the water-in-oil type, of additives which ensure that the rheological properties of the drilling fluid do not change even when increasing quantities of free carboxylic acids are released as a result of partial ester hydrolysis. Where possible, these free carboxylic acids should be transferred to compounds displaying stabilizing and emulsifying properties. For this purpose, EP 0 382 701 A1 proposes adding highly oleophilic alkaline amines which display as low water solubility as possible and can form salts with the free acids. Typical examples of amine compounds of this type are primary, secondary and/or tertiary amines which are predominantly water-insoluble and which can in addition be at least partially alkoxylated and/or substituted with hydroxyl groups. Further examples include amino amides and/or heterocycles containing nitrogen as the ring atom.

Basic amines comprising at least one long-chain hydrocarbon radical containing 8 to 36 carbon atoms, preferably containing 10 to 24 carbon atoms, are for example suitable, wherein these hydrocarbon radicals can also be singly or multiply unsaturated.

The amounts in which the further additives described hereinbefore of the composition used as a drilling fluid are added, in the case of a water-in-oil emulsion, conventionally correspond to those amounts in which these compounds are added to the water-in-oil-based drilling fluids known in the art.

In low-weighted compositions, component IV) is preferably a weighting agent, such as for example BaSO₄, component IV) being used preferably in an amount of up to 20% by weight in the case of a low-weighted composition. In more highly weighted compositions, component IV) is used preferably in an amount of from 20 to 50% by weight, whereas 50 to 70% by weight of component IV) can be used in highly weighted compositions.

Furthermore, it is preferable in accordance with the invention for the composition, in so far as it is in the form of a water-in-oil emulsion, to be a nanoemulsion or a microemulsion which preferably contains drops of water or drops of an aqueous phase having a drop size of less than 1,000 μm, preferably having a drop size in a range of from 5 nm to 1,000 μm, particularly preferably having a drop size in a range of from 10 nm to 850 μm, even more preferably having a drop size in a range of from 20 nm to 700 μm, even more preferably having a drop size in a range of from 50 nm to 500 μm. According to the invention, the terms “microemulsion” and “nanoemulsion” denote emulsions containing drops in the micrometer or nanometer range, wherein there can be a certain degree of overlap of these two ranges and thus also of these two terms. According to a portion of the specialist literature and also of the prior art relating to drilling fluids, the term “microemulsions” preferably refers to emulsions of the type that are formed spontaneously when the emulsion components are combined, whereas the formation of nanoemulsions conventionally requires the supplying of energy, for example in the form of homogenization, in particular in the form of high-pressure homogenization.

In the case of a water-in-oil emulsion as a composition used as a drilling fluid, the emulsion can be produced by any method known to the person skilled in the art for producing a water-in-oil emulsion of this type. Thus, it is in particular conceivable firstly to produce the base emulsion from the organic oil phase as a continuous phase and the drops of water emulsifying therein and only then to add the n-nonyl ether described at the outset and if appropriate the further additives. However, it is also conceivable firstly to add the n-nonyl ethers described at the outset to the organic oil phase and then to form the emulsion from this oil phase and the water or the aqueous solution.

According to another particular embodiment of the composition used as a drilling fluid, the composition is an aqueous solution or an oil-in-water emulsion.

In this connection, it is particularly preferable for the composition to contain
I) 0 to 48% by weight, particularly preferably 0.1 to 20% by weight and most preferably 1 to 10% by weight, based in each case on the total weight of the composition, of an organic oil phase which is not miscible with water,
II) 29.9 to 99.9% by weight, particularly preferably 60 to 99% by weight and most preferably 70 to 95% by weight, based in each case on the total weight of the composition, of water or aqueous phase,
III) 0.1 to 20% by weight, particularly preferably 1 to 15% by weight and most preferably 5 to 10% by weight, based in each case on the total weight of the composition, of the n-nonyl ether described at the outset,
IV) 0 to 70% by weight, particularly preferably 1 to 5% by weight and most preferably 1.5 to 3% by weight, based in each case on the total weight of the composition, of at least one further additive,
   the sum of components I) to IV) being 100% by weight.

The organic oil phase, aqueous phase and further additives are preferably those organic oil phases, aqueous phases and further additives which were mentioned at the outset in relation to the water-in-oil emulsion.

Even in the case of an oil-in-water emulsion as a composition used as a drilling fluid, the composition can be produced by any method known to the person skilled in the art for producing an oil-in-water emulsion of this type. Thus, it is in particular conceivable firstly to produce the base emulsion from water or the aqueous solution as a continuous phase and the drops emulsified therein of the oil phase and only then to add the n-nonyl ether described at the outset and if appropriate the further additives. However, it is also conceivable firstly to add the n-nonyl ethers described at the outset to the organic oil phase and then to form the emulsion from this oil phase and the water or the aqueous solution.

According to a preferred embodiment of this method for producing boreholes, in which a drilling fluid is pumped through a borehole, the method comprises the method steps:
(α1) providing the composition according to the invention, in particular the composition according to the invention in the form of a water-in-oil emulsion, an aqueous solution or an oil-in-water emulsion;
(α2) drilling a hole into the ground;
(α3) introducing, preferably circulating, the composition provided in method step (α1) at least partially into and/or in the borehole;
   the introducing, preferably the circulating, taking place preferably at least partially during the drilling in method step (α2).

The composition according to the invention therefore acts as a drilling fluid during the drilling of holes into the ground, preferably during the drilling for crude oil or natural gas.

A contribution to achieving the objects mentioned at the outset is therefore also provided by a method for producing an oil or a gas, comprising the method steps:
(α1) providing the composition used as a drilling fluid, in particular the composition used as the drilling fluid in the form of a water-in-oil emulsion, an aqueous solution or an oil-in-water emulsion;
(α2) drilling a hole into the ground;
(α3) introducing, preferably circulating, the composition provided in method step (α1) at least partially into and/or in the borehole, introducing or circulating taking place, in this case too, preferably at least partially during the drilling in method step (α2);
(α4) conveying oil or gas out of the ground through the hole drilled in method step (α2);
(α5) optionally purifying or refining the oil or gas conveyed in method step (α3).

A contribution to achieving the objects mentioned at the outset is also made by a cleaning agent and also a drilling fluid, preferably a drilling fluid in the form of the water-in-oil emulsion described hereinbefore or the oil-in-water emulsion described hereinbefore.

The invention will now be explained in greater detail based on non-limiting examples.

### EXAMPLE 1

### Production of Di-n-Nonyl Ether

31.6 g of pelargonic acid (0.2 mol, Emery® 1203) and 150 ml of methanol were placed in a glass flask and mixed with 3 g of concentrated sulphuric acid. The mixture was heated to boiling for 4 hours under reflux. Afterwards, 3.5 g of anhydrous sodium carbonate were added and the excess alcohol was removed by distillation. The pelargonic acid methyl ester was removed by distillation under vacuum (p approx. 16 mbar) at 95-100° C.

29.2 g of the pelargonic acid methyl ester obtained in this way were mixed with 6% by weight of copper chromite catalyst and stirred for 4 hours in an autoclave at 230° C. and a hydrogen pressure of 250 bar. Afterwards, the catalyst was filtered off and the filtrate was distilled under vacuum. The boiling point was about 113° C. at 26 mbar, the yield was 79%.

The approach described hereinbefore for producing n-nonanol was repeated several times.

577 g of the n-nonanol obtained in this way were placed in a flask with a water separator and mixed with 0.1 g of trifluormethanesulphonic acid. The reaction mixture was heated under reflux (approx. 225° C.) until 36 ml of water had precipitated. After cooling, the product is washed with 100 ml of 5% sodium hydroxide solution and 100 ml of water. For working-up, the crude product was dried and distilled under vacuum. Di-n-nonyl ether was obtained at a yield of 503 g.

### EXAMPLE 2

### Production of Nonyl Diethylene Glycol Ether

433 g of n-nonyl alcohol, which was obtained as in Example 1 by reduction of pelargonic acid methyl ester, were mixed with 5 g of a 30% by weight solution of potassium hydroxide in methanol and heated in an autoclave to 100° C. At this temperature, the traces of methanol which were present were removed by evacuating five times and aerating with nitrogen. After increasing the reaction temperature to 150° C., a total of 264 g of ethylene oxide were added in portions, so that the pressure in the reactor did not exceed 5×10⁵ Pa. After completion of the reaction, the mixture was cooled to about 90° C. and evacuated for approx. 15 minutes for separating off remaining traces of ethylene oxide. A light yellow liquid was obtained.

### EXAMPLE 3

### Production of a Thermoplastic Composition

6 kg of polyethylene terephthalate (PET SP04 from the company Catalana de Polimers) are introduced in a 15 kg Henschel mixer. The mixing wall temperature was 40° C. Furthermore, 0.3% by weight of the di-n-nonyl ether produced in Example 1 were added as a mold release agent. Subsequently, the material was granulated on a granulator (ZSK 26Mcc) with a stuffing screw.

A Battenfeld HM800/210-type fully hydraulic injection molding machine with a hydraulic closing unit was used for producing molded articles from the thermoplastic composition. The maximum closing force is 800 kN, the screw diameter is 25 mm. A mold having a conically tapering, rectangular core was used as the test mold. A force transducer having a maximum measuring range of 2 kN was attached to the ejector rod for determining the demolding force. The molding compound was predried at about 225° C. for about 4 hours. With the thermoplastic composition according to the invention, demolding was observed that was greatly improved over a mold release agent-free molding compound.

### EXAMPLE 4

### Production of a Washing Agent

0.2% by weight of zinc ricinoleate (Tego® Sorb Conc 50 from Goldschmidt), 1% by weight of sodium citrate, 0.1% by weight of the di-n-nonyl ether obtained in Example 1 as the defoamer, 1% by weight of boric acid, 7.5% by weight of glycerol, 1% by weight of ethanol, 4% by weight of C₁₂-C₁₆ alkylglycoside, 8% by weight of soap, 8% by weight of C₁₂-C₁₄ fatty alcohol+1.3 EO sulphate sodium salt, 1% by weight of Acusol 120 (15%; methacrylic acid (stearyl alcohol 20 EO) ester-acrylic acid copolymer from Rohm & Haas), 0.5% by weight of Dequest 2066, amylase, protease, and also water were mixed, a washing agent being obtained.

### EXAMPLE 5

### Production of an Adhesive

According to the teaching of DE-A-199 57 351, a high-molecular diisocyanate was produced from a polypropylene glycol, wherein Mn=880, and diphenylmethane diisocyanate, from which high-molecular diisocyanate the monomeric MDI was subsequently removed to the extent that a residual monomer content of 0.1% resulted. A hot-melt adhesive was produced from 100 parts of a polyol mixture for a standard polyurethane hot-melt adhesive (QR 6202, company Henkel®) having an averaged OH number of 32.5 and 76.5 parts of the aforementioned high-molecular diisocyanate. 5% by weight of the nonyl diethylene glycol ether produced in Example 2 were additionally added.

### EXAMPLE 6

### Production of a Defoamer

4.0% by weight of paraffin having a solidification point in accordance with DIN ISO 2207 of 45° C., a liquid content at 40° C. of about 66% by weight and a liquid content at 60° C. of about 96% by weight, 1.2% by weight of bisamide, 3% by weight of sodium carbonate, 58.7% by weight of sodium sulphate, 21.4% by weight of sodium silicate, 2.1% by weight of cellulose ether, 4.8% by weight of the di-n-nonyl ether obtained in Example 1 and water are mixed so as to form an aqueous slurry which was spray-dried with superheated steam in accordance with the method of European patent specification EP 625 922.

### EXAMPLE 7

### Production of an N-Nonyl Ether-Based Defoamer

1.2% by weight of bisamide, 3% by weight of sodium carbonate, 58.7% by weight of sodium sulphate, 21.4% by weight of sodium silicate, 2.1% by weight of cellulose ether, 8.8% by weight of the di-n-nonyl ether obtained in Example 1 and water are mixed so as to form an aqueous slurry which was spray-dried with superheated steam in accordance with the method of European patent specification EP-A-0 625 922.

### EXAMPLE 8

### Production of a Textile Auxiliary

5 g of the polymer emulsion produced in accordance with Example 1b of DE-A-39 39 549 were added to 995 g of a textile lubricant, consisting of 78.5% by weight of i-butyl stearate, 5% by weight of oleyl/cetyl alcohol 5 mol EO, 2.2% by weight of coconut fatty acid monoethanolamide 4 mol EO, 0.8% by weight of oleic acid, 6% by weight of the nonyl diethylene glycol ether obtained in Example 2, 6% by weight of secondary fatty alcohol 7 mol EO (Tergitol 15S7, manufacturer: Union Carbide®) and 1.5% by weight of water, at 20° C. while stirring (maximum stirring speed of a head stirrer with a propeller stirrer). After 30 seconds the polymer emulsion had been distributed uniformly and a clear solution had been formed. Afterwards, the stirring speed was reduced as far as possible and the textile lubricant was heated to 60° C. in order to speed up the decomposition of the polymer particles.

### EXAMPLE 9

### Production of a Paint

736 g of demineralized water, 4 g of a 70% by weight solution of stearic acid isodecyl ester in C₁₂H₂₆ (isomer mix), 10 g of nitrobenzenesulphonic acid sodium, 5 g of tetrasodium salt of ethylenediaminetetraacetic acid, 100 g of urea, 25 g of sodium bicarbonate, 100 g of D-I.1, 20 g of fluorescent brightener C.I. 230 were put in place. 5 g of the di-n-nonyl ether obtained in Example 1 were added as a defoamer and the mixture was stirred for 60 seconds with a high-speed stirrer at 2,000 rpm.

### EXAMPLE 10

### Production of a Cosmetic Formulation

O/W emulsions were produced, the oil phases of which had the following composition:
5.0 g of the compounds characterized in EP-A-1 485 061 by Formula (I) in which R′ represents methyl and R represents in each case a butyloctanoyl radical (C₁₂),
5.0 g of dioctyl ether emulsifier (Cetiol OE, company Cognis®),
0.6 g of cetyl stearyl alcohol emulsifier+20-EO (Eumulgin B2, company Cognis®),
0.1 g of creatine.

5% by weight of the nonyl diethylene glycol ether obtained in Example 2 were added to the composition obtained in this way.

### EXAMPLE 11

### Production of a Drilling Fluid

A conventional lime fluid was produced from 7.6 g of prehydrated bentonite, 1.15 g of ferrochrome lignosulphonate, 2.3 g of slaked lime, 0.38 g of starch and 0.76 g of NaOH. 5% by weight of the nonyl diethylene glycol ether obtained in Example 2 were added to this lime fluid.

1. A method for producing an organic composition, comprising as method steps:
i) providing
ia) an n-nonyl ether as an additive that can be obtained by reacting an n-nonyl alcohol component with a further component which is able to react with the n-nonyl alcohol component so as to form an n-nonyl ether,
ib) a functional component, and also optionally
ic) at least one further additive;
ii) mixing the n-nonyl ether, the functional component and optionally the at least one further additive;
wherein said functional component is selected from a thermoplastic polymer, an enzyme, a setting agent, a paraffin, an oil, a colorant and a hair care or skin care substance.

2. The method according to claim 1, wherein the further component is able to react with the n-nonyl alcohol component so as to form an n-nonyl ether is an alcohol, an epoxide, a halogen alkane or a mixture of at least two thereof.

3. The method according to claim 2, wherein the further component is an alcohol, selected from the group consisting of C₁ to C₃₀ alkanols, C₁ to C₃₀ diols, C₁ to C₃₀ triols, polyalcohols, polyether alcohols and mixtures of at least two of these alcohols.

4. The method according to claim 1, wherein the n-nonyl ether is present as a modified n-nonyl ether in the form of an organic or inorganic ester.

5. The method according to claim 1, wherein the further additive is used in an amount in a range of from about 0.001 to about 40% by weight, based on the composition.

6. The method according to claim 1, wherein at least about 80% by weight of the n-nonyl alcohol component, based on the n-nonyl alcohol component, is obtained from pelargonic acid.

7. The method according to claim 6, wherein the pelargonic acid is obtained from oleic acid.

8. The method according to claim 7, wherein the pelargonic acid is produced petrochemically.

9. The method according to claim 1, wherein the n-nonyl alcohol component contains less than about 10% by weight, based on the n-nonyl alcohol component, of C₈ and C₁₀ alcohols.

10. The method according to claim 1, wherein the further component is a polyether alcohol containing 2 to 30 ether repeating units or epoxide.

11. The method according to claim 1, wherein the functional component is a thermoplastic polymer and the composition obtained is a thermoplastic composition.

12. A method for producing a shaped article, comprising the method steps:
I) providing a thermoplastic composition which can be obtained using the method according to claim 11;
II) heating the thermoplastic composition to the glass transition temperature of the thermoplastic polymer or to a temperature above the glass transition temperature of the thermoplastic polymer;
III) producing a shaped article from the heated, thermoplastic composition produced in method step II).

13. The method according to claim 12, wherein, in a further method step IV), at least a partial region of the shaped article obtained in method step III) is reduced in its mass cross section in relation to method step III).

14. The method according to claim 12, wherein the shaped article is selected from a group consisting of a vessel, a foil, a fiber or at least two thereof.

15. A method for producing an item to be packaged comprising as method steps:
a) providing an item and a shaped article which can be obtained using a method according to claim 12;
b) at least partially surrounding the item with the shaped article.

16. A method for coating substances which can be consumed by living beings, comprising as method steps:
A) providing a substance which can be consumed by living beings;
B) providing an n-nonyl ether which can be obtained by reacting an n-nonyl alcohol component with a further component which is able to react with the n-nonyl alcohol component so as to form an n-nonyl ether; and
C) at least partially surrounding the substance which can be consumed by living beings with the n-nonyl ether.

17. A use of at least one n-nonyl ether which can be obtained by reacting an n-nonyl alcohol component with a further component which is able to react with the n-nonyl alcohol component so as to form an n-nonyl ether, as additive in a composition comprising as a functional component
α) a thermoplastic polymer, wherein the composition is a thermoplastic composition;
β) an enzyme, wherein the composition is a washing agent;
γ) a setting agent of an adhesive, wherein the composition is an adhesive;
δ) a paraffin, wherein the component is a defoamer;
ε) an oil, wherein the composition is a lubricant formulation;
ζ) a colorant, wherein the composition is a paint or a dye; or
η) a hair care or skin care substance, wherein the composition is a cosmetic preparation.

18-23. (canceled)

24. A use of an n-nonyl ether which can be obtained by reacting an n-nonyl alcohol component with a further component which is able to react with the n-nonyl alcohol component so as to form an n-nonyl ether, as an additive in compositions used in the drilling of boreholes.

25. (canceled)

26. The use according to claim 24, wherein the further component which is able to react with the n-nonyl alcohol component so as to form an n-nonyl ether is an alcohol, an epoxide, a halogen alkane or a mixture of at least two thereof

27. The use according to claim 26, wherein the further component is an alcohol, selected from the group consisting of C₁ to C₃₀ alkanols, C₁ to C₃₀ diols, C₁ to C₃₀ triols, polyalcohols, polyether alcohols and mixtures of at least two of these alcohols.

28-32. (canceled)

33. The method for cleaning the surfaces of boreholes, drilling devices or drill cuttings, comprising the method steps:
(β1) drilling a borehole into the ground by means of a drill head driven via a drill rod assembly,
(β2) introducing a casing into the borehole, and
(β3) introducing cement into at least a partial region of the intermediate space between the outer side of the casing and the walls of the borehole,
wherein, before method step (β3) is carried out, the cleaning agent comprising an n-nonyl ether, as defined in claim 24, obtained by reacting an n-nonyl alcohol component with a further component which is able to react with the n-nonyl alcohol component so as to form an n-nonyl ether, as an additive in compositions used in the drilling of boreholes is passed through the intermediate space between the outer side of the casing and the walls of the borehole.

34. The method according to claim 32, wherein the cleaning agent comprising an n-nonyl ether, is at least partially passed through the drill head during step (β1), this passing-through being carried out at least partially while the drill head is present in the borehole.

35. A method for producing a borehole, comprising the method steps
(β1) drilling a borehole into the ground by means of a drill head driven via a drill rod assembly,
(β2) introducing a casing into the borehole,
(β3) introducing cement into at least a partial region of the intermediate space between the outer side of the casing and the walls of the borehole,
(β4) optionally introducing a conveyor pipe into the casing,
(β5) optionally introducing a sealing liquid into the intermediate space between the outer side of the conveyor pipe and that of the inner side of the casing,
wherein surfaces of the borehole, the guide pipe, the drill rod assembly or the drill head are brought into contact with a cleaning agent comprising an n-nonyl ether, as defined in claim 24.

36. A method for producing an oil or a gas, comprising the method steps
(β1) drilling a borehole into the ground by means of a drill head driven via a drill rod assembly,
(β2) introducing a casing into the borehole,
(β3) introducing cement into at least a partial region of the intermediate space between the outer side of the casing and the walls of the borehole,
(β4) optionally introducing a conveyor pipe into the casing,
(β5) optionally introducing a sealing liquid into the intermediate space between the outer side of the conveyor pipe and that of the inner side of the casing,
(β6) conveying oil or gas through the borehole, and also
(β7) purifying or refining the conveyed oil or gas,
wherein surfaces of the borehole, the guide pipe, the drill rod assembly or the drill head are brought into contact with a cleaning agent comprising an n-nonyl ether, as defined in claim 24, or with a composition, comprising an n-nonyl ether, obtained by reacting an n-nonyl alcohol component with a further component which is able to react with the n-nonyl alcohol component so as to form an n-nonyl ether, as an additive in compositions used in the drilling of boreholes.

37. A method for producing boreholes, in which a drilling fluid is pumped through a borehole, wherein a composition comprising an n-nonyl ether, as defined in claim 24, is used as the drilling fluid.

38. (canceled)

39. Method according to claim 37, wherein the composition contains:
I) about 28.9 to about 99% by weight, based on the total weight of the composition, of an organic oil phase which is not miscible with water,
II) about 1 to about 48% by weight, based on the total weight of the composition, of water or aqueous phase,
III) about 0.1 to about 20% by weight, based on the total weight of the composition, of the n-nonyl ether defined in claims 24 and 26 to 31,
IV) 0 to about 70% by weight, based on the total weight of the composition, of at least one further additive, the sum of components I) to IV) being 100% by weight;
wherein said composition is a water-in-oil emulsion.

40. The method according to claim 39, wherein the water-in-oil emulsion is a nanoemulsion or a microemulsion comprising drops of water or drops of an aqueous phase having a drop size in a range of from 5 nm to 1,000 μm.

41. (canceled)

42. The method according to claim 41, wherein the composition contains:
I) 0 to about 48% by weight, based on the total weight of the composition, of an organic oil phase which is not miscible with water,
II) about 29.9 to about 99.9% by weight, based on the total weight of the composition, of water or aqueous phase,
III) about 0.1 to about 20% by weight, based on the total weight of the composition, of the n-nonyl ether defined in claims 24 and 26 to 31,
IV) 0 to about 70% by weight, based on the total weight of the composition, of at least one further additive,
the sum of components I) to IV) being 100% by weight;
wherein said composition is an aqueous solution or an oil-in-water emulsion.

43. The method according to claim 39, wherein the at least one further additive is an additive selected from the group consisting of thickening agents, clays, liquid loss prevention agents, pH modifiers, viscosity modifiers, filtration control agents, emulsifiers, salts, wetting agents, weighting agents and dispersants.

44. The method according to claim 39, comprising the method steps:
(α1) providing said composition;
(α2) drilling a hole into the ground; and
(α3) introducing said, composition at least partially into and/or in the borehole.

45. (canceled)

46. A method for producing an oil or a gas, comprising the method steps
(α1) providing a composition according to claim 35;
(α2) drilling a hole into the ground;
(α3) introducing, the composition provided in method step (α1) at least partially into and/or in the borehole;
(α4) conveying oil or gas out of the ground through the hole drilled in method step (α2);
(α5) optionally purifying or refining the oil or gas conveyed in method step (α3).
